(19) United States
(12) Patent Application Publication
(10) Pub. No.: US 2010/0268098 A1
(43) Pub. Date: Oct. 21, 2010

(54) SPHYGMOMANOMETER AND MEASUREMENT ACCURACY CHECK SYSTEM OF SPHYGMOMANOMETER

(75) Inventors: Shinichi Ito, Kyoto (JP); Yukiya Sawanoi, Nara (JP); Shingo Yamashita, Kyoto (JP); Mika Eto, Osaka (JP); Takahide Tanaka, Shiga (JP)

Correspondence Address: OSHA LIANG L.L.P., TWO HOUSTON CENTER, 909 FANNIN, SUITE 3500, HOUSTON, TX, 77010, US

(73) Assignee: OMRON HEALTHCARE CO., LTD., Kyoto (JP)

(21) Appl. No.: 12/810,680

(22) PCT Filed: Jan. 15, 2009
(86) PCT No.: PCT/JP2009/050457

§ 371 Date: Jun. 25, 2010

(30) Foreign Application Priority Data
Jan. 23, 2008 (JP) 2008-012843

(51) Int. Cl. A61B 5/02

(52) U.S. Cl. 600/490

(57) ABSTRACT

A measurement accuracy check system of a sphygmomanometer includes a sphygmomanometer having a blood pressure measurement mode for measuring a blood pressure based on a change in internal pressure of a cuff attached to a blood pressure measurement site, and an accuracy check mode for checking measurement accuracy in the blood pressure measurement mode, and an accuracy check device communicably connected with the sphygmomanometer for determining the measurement accuracy of the sphygmomanometer in the accuracy check mode. The sphygmomanometer includes an air system piping communicating to the cuff in the blood pressure measurement mode and communicating to an air system of the accuracy check device in the accuracy check mode, a pressurization and depressurization unit for adjusting pressure to be applied to the air system piping, and a first pressure detection unit for detecting pressure in the air system piping.

### TECHNICAL FIELD

The present invention relates to a sphygmomanometer, and a measurement accuracy check system for checking measurement accuracy of the sphygmomanometer.

### BACKGROUND ART

In recent years, the lifestyle-related diseases caused by high blood pressure have been becoming common, and daily measurement and management of the blood pressure value are important as an index of daily health management. Thus, a home sphygmomanometer is being widespreadly used.

If drawbacks such as incapability in measurement of the blood pressure value occur in the home sphygmomanometer, a user generally sends the sphygmomanometer to the manufacturing company for inspection and repair services.

However, if the blood pressure value measured at home greatly deviates from a user predicted blood pressure value or if the blood pressure value measured at home does not match a blood pressure value measured at a medical institution even in a case where drawbacks such as measurement incapability have not occurred, many users are concerned about measurement accuracy of the sphygmomanometer.

In such a case, since the blood pressure itself easily fluctuates by living environment and stress, whether the deviation of the blood pressure value is due to lowering of the measurement accuracy of the sphygmomanometer or due to the fluctuation in the blood pressure is difficult for the user to determine.

Thus, when the user sends the sphygmomanometer to the manufacturing company to receive the inspection service on the measurement accuracy, there is a period in which the blood pressure values cannot be measured. Furthermore, there is a state in which the sphygmomanometer is continuously used while feeling insecurity in the measurement accuracy as it is troublesome to send the sphygmomanometer to the manufacturing company.

With regards to the measurement accuracy of a sphygmomanometer, Japanese Unexamined Patent Publication No. 7-51233 (patent document 1) discloses an electronic sphygmomanometer that obtains pressure adjustment data (e.g., sensitivity coefficient or linear correction data) unique to a measuring instrument in time of production, and sets and stores the same in non-volatile storage means. Thus productivity and accuracy can be enhanced since there are not required tasks such as the human adjusts the half-fixed resistor, performs pattern cutting of a substrate, and the like for every measuring instrument.

Patent document 1: Japanese Unexamined Patent Publication No. 7-51233

### DISCLOSURE OF THE INVENTION

### Problems to be Solved by the Invention

In the electronic sphygmomanometer described in Japanese Unexamined Patent Publication No. 7-51233, there is disclosed a configuration of correcting a cuff pressure detected with a pressure sensor in time of measurement using pressure adjustment data in time of production set and stored in the non-volatile storage means. However, the sensitivity coefficient and the linearity of the pressure sensor are variable values corresponding to the usage environment such as the usage period of the measuring instrument, and gradually differ from the pressure adjustment data in time of production with elapse of the usage period, so that the measurement accuracy of the electronic sphygmomanometer cannot necessarily be guaranteed. The insecurity felt by the user with respect to the measurement accuracy of the electronic sphygmomanometer thus cannot be resolved. Japanese Unexamined Patent Publication No. 7-51233 does not disclose any means for the user to check the measurement accuracy of the electronic sphygmomanometer.

There is used in medical institutions and the like a sphygmomanometer having a configuration in which two pressure sensors are built in a main body of a sphygmomanometer and the measurement accuracy of the sphygmomanometer is determined based on pressure deviation between pressure values detected by the respective pressure sensors to check the measurement accuracy of the sphygmomanometer in time of use.

However, such a configuration has a problem that application to a home sphygmomanometer is not easy as the main body of the sphygmomanometer enlarges and the device cost increases due to mounting of two pressure sensors.

In view of solving the above problems, it is an object of the present invention to provide a sphygmomanometer having an easy and inexpensive device configuration and capable of checking measurement accuracy, as well as a measurement accuracy cheek system of the sphygmomanometer.

### Means for Solving the Problem

A measurement accuracy check system of a sphygmomanometer according to one aspect of the present invention includes: a sphygmomanometer having a blood pressure measurement mode for measuring a blood pressure based on a change in internal pressure of a cuff attached to a blood pressure measurement site, and an accuracy check mode for checking measurement accuracy in the blood pressure measurement mode; and an accuracy check device communicably connected with the sphygmomanometer for determining the measurement accuracy of the sphygmomanometer in the accuracy check mode, wherein the sphygmomanometer includes: an air system piping communicating to the cuff in the blood pressure measurement mode and communicating to an air system of the accuracy check device in the accuracy check mode; a pressurization and depressurization unit for adjusting pressure to be applied to the air system piping; and a first pressure detection unit for detecting pressure in the air system piping, the accuracy check device includes: a pressure generator for generating pressure in the air system according to a predetermined pressure generation pattern set in advance; and a second pressure detection unit for detecting pressure in the air system, and one of the sphygmomanometer and the accuracy check device includes: a measurement accuracy determining portion for determining the measurement accuracy of the sphygmomanometer based on a difference value between a pressure detection value of the first pressure detection unit and a pressure detection value of the second pressure detection unit; and a display unit for displaying the determined measurement accuracy of the sphygmomanometer.

The predetermined pressure generation pattern preferably includes a pulse wave generation pattern for expressing a change in pulse pressure detected by the first pressure detection unit in the blood pressure measurement mode.

Preferably, the predetermined pressure generation pattern further includes a generation pattern for applying pressure to the air system piping for a predetermined period set in advance, and the measurement accuracy determining portion includes an operation performance diagnosis portion for diagnosing operation performance of a component of the pressurization and depressurization unit based on the pressure detection value of the first pressure detection unit after elapse of the predetermined period.

Preferably, the sphygmomanometer further includes: a storage unit for storing the determined measurement accuracy of the sphygmomanometer in association with check date and time of the measurement accuracy; and a notifying unit for notifying whether or not the blood pressure measurement mode is executable on the user based on the measurement accuracy stored in the storage unit.

The notifying unit preferably notifies to urge execution of the accuracy check mode to the user in association with the notification.

Preferably, the sphygmomanometer further includes an operation unit for outputting a signal for instructing selection of the accuracy check mode in response to an operation by the user.

Preferably, the sphygmomanometer further includes a connector for coupling the air system piping and an air system of the accuracy check device, the accuracy check mode being selected in response to closing of the connector.

A sphygmomanometer according to another aspect of the present invention has a blood pressure measurement mode for measuring a blood pressure based on a change in internal pressure of a cuff attached to a blood pressure measurement site, and an accuracy check mode for checking measurement accuracy in the blood pressure measurement mode. The sphygmomanometer includes: an air system piping communicating to the cuff in the blood pressure measurement mode; a pressurization and depressurization unit for adjusting pressure to be applied to the air system piping; a first pressure detection unit for detecting pressure in the air system piping, the air system piping communicating to an air system of an accuracy check device arranged outside the sphygmomanometer in the accuracy check mode, and being applied with pressure having a predetermined pressure generation pattern generated in the air system by the accuracy check device; a measurement accuracy determining portion for determining the measurement accuracy of the sphygmomanometer based on a difference value between a pressure detection value of the first pressure detection unit and a predetermined pressure reference value set in advance in the accuracy check mode; and a display unit for displaying the determined measurement accuracy of the sphygmomanometer.

Preferably, the sphygmomanometer further includes a mode selection unit for detecting a pressure signal of the air system of the accuracy check device and selecting the accuracy check mode.

### Effects of the Invention

According to the present invention, the measurement accuracy of the sphygmomanometer can be checked with a simple and inexpensive device configuration. As a result, the user can perform measurement of the daily blood pressure values with the stable measurement accuracy.

### BRIEF DESCRIPTION OF THE DRAWINGS

FIG. 1 is a schematic view of an outer appearance of a measurement accuracy check system of a sphygmomanometer according to a first embodiment of the present invention.

FIG. 2 is a block diagram showing a specific example of a hardware configuration of the sphygmomanometer 1 and an accuracy check device 60.

FIG. 3 is a block diagram showing a specific example of a functional configuration for performing a check operation of measurement accuracy of the sphygmomanometer 1.

FIG. 4 is a view for describing a pseudo-pulse wave generated by a pressure generator 84.

FIG. 5 is a view for describing a change in internal pressure of an air tube 90 detected with a pressure sensor 20.

FIG. 6 is a view showing a display example on a display unit 4.

FIG. 7 is a view showing another display example on the display unit 4.

FIG. 8 is a view showing another display example on the display unit 4.

FIG. 9 is a flowchart for describing a measurement accuracy check operation of the sphygmomanometer 1, executed by a CPU 50 of the sphygmomanometer 1 and a CPU 80 of the accuracy check device 60.

FIG. 10 is a view showing a pressure generation pattern when diagnosing an airtightness of a measurement air system 23.

FIG. 11 is a flowchart for describing a diagnosis operation of the airtightness of the measurement air system 23 in the sphygmomanometer 1, executed by the CPU 50 of the sphygmomanometer 1 and the CPU 80 of the accuracy check device 60.

FIG. 12 is a view showing a pressure generation pattern when diagnosing dynamic characteristics of the pressure sensor 20.

FIG. 13 is a view showing a pressure generation pattern when diagnosing dynamic characteristics of a pump 24.

FIG. 14 is a flowchart for describing the diagnosis operation of the dynamic characteristics of the pump 24 in the sphygmomanometer 1, executed by the CPU 50 of the sphygmomanometer 1 and the CPU 80 of the accuracy check device 60.

FIG. 15 is a view showing a pressure generation pattern when diagnosing dynamic characteristics of a valve 28.

FIG. 16 is a flowchart for describing the diagnosis operation of the dynamic characteristics of the valve 28 in the sphygmomanometer 1, executed by the CPU 50 of the sphygmomanometer 1 and the CPU 80 of the accuracy check device 60.

FIG. 17 is a block diagram showing a specific example of a functional configuration for performing a check operation of measurement accuracy of a sphygmomanometer 1 in a measurement accuracy check system of the sphygmomanometer according to a variant of the first embodiment.

FIG. 18 is a view showing a display example on the display unit 4

FIG. 19 is a schematic view of an outer appearance of a measurement accuracy check system of a sphygmomanometer according to a second embodiment of the present invention.

FIG. 20 is a block diagram showing a specific example of a hardware configuration of a sphygmomanometer 1A and an accuracy check device 60A.

FIG. 21 is a block diagram showing a specific example of a functional configuration for performing a check operation of measurement accuracy of the sphygmomanometer 1A.

FIG. 22 is a flowchart for describing the measurement accuracy check operation of the sphygmomanometer 1A, executed by a CPU 50A of the sphygmomanometer 1A and a CPU 80A of the accuracy check device 60A.

### DESCRIPTION OF SYMBOLS

1, 1A sphygmomanometer
2, 2A main body
3, 3A, 64, 64A operation unit
4, 4A, 62, 62A display unit
5 cuff
6 connector
8 air bladder
10, 90 air tube
20, 82 pressure sensor
22 A/D converter
23 measurement air system
24 pump
26, 30 drive circuit
28 valve
40 to 48, 480 display region
52, 88 memory
54, 86 timer
60, 60A accuracy check device
70 communication line
84 pressure generator
92 connection plug
302, 642 power switch
304 measurement switch
306 accuracy check mode switch
308, 646 diagnosis item switch
502 accuracy check mode setting portion
504, 802 pressure measuring portion
506 measurement accuracy managing portion
508, 806 display processing portion
512 measurement accuracy determining portion
648 pressurization switch
804 measurement accuracy determining portion

### BEST MODE FOR CARRYING OUT THE INVENTION

Embodiments of the present invention will be described below in detail with reference to the drawings. The same symbols are denoted for the same or corresponding portions in the drawings.

### First Embodiment

### (Configuration of Measurement Accuracy Check System of Sphygmomanometer)

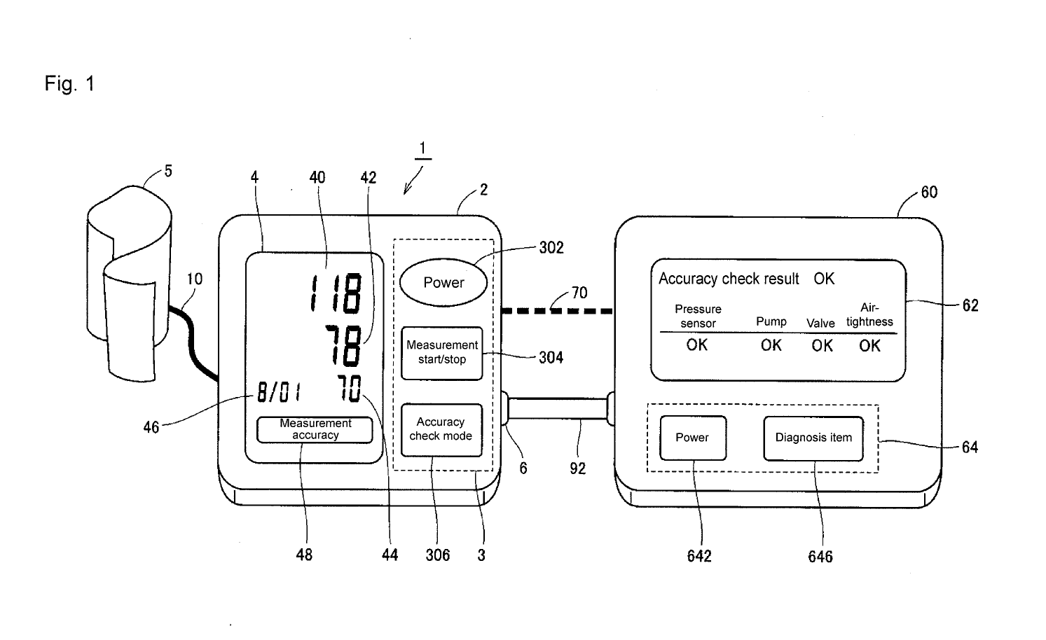

FIG. 1 is a schematic view of an outer appearance of a measurement accuracy check system of a sphygmomanometer according to a first embodiment of the present invention.

With reference to FIG. 1, the measurement accuracy check system of the sphygmomanometer includes a sphygmomanometer 1, an accuracy check device 60 for checking measurement accuracy of the sphygmomanometer 1, an accuracy check device connection plug (hereinafter referred to as connection plug) 92, and a communication line 70.

When performing a check operation of the measurement accuracy with respect to the sphygmomanometer 1, the connection plug 92 is coupled to a connector 6 arranged in a main body 2 of the sphygmomanometer 1, and the communication line 70 is arranged between the main body 2 and the accuracy check device 60.

The sphygmomanometer 1 includes the main body 2 and a cuff 5 to be wrapped around an upper arm as a measurement site, which are connected with each other by an air tube 10. An operation unit 3 such as a switch and a display unit 4 for displaying a measurement result are arranged on a front surface of the main body 2.

The operation unit 3 includes a power switch 302 for instructing ON/OFF of the power supply, a measurement switch 304 for instructing start/stop of the measurement, and a switch (hereinafter referred to as accuracy check mode switch) 306 for instructing selection of “accuracy check mode”.

The “accuracy check mode” is an operation mode for checking the measurement accuracy of the sphygmomanometer 1. The sphygmomanometer 1 includes a “blood pressure measurement mode” for performing the normal blood pressure measurement operation, and the accuracy check mode as operation modes. The sphygmomanometer 1 transitions from the blood pressure measurement mode to the accuracy check mode when receiving input of an operation signal by the operation of the accuracy check mode switch 306.

The display unit 4 includes display regions 40 to 46 for displaying the measurement result. The display regions 40 to 44 show systolic blood pressure data indicating the systolic blood pressure, diastolic blood pressure data indicating the diastolic blood pressure, and number of pulse data indicating the number of pulses. The display region 46 displays time data indicating the date and time of the blood pressure measurement.

The display unit 4 also includes a display region 48 for displaying measurement accuracy data indicating the measurement accuracy of the sphygmomanometer 1 at the date and time of the blood pressure measurement. The measurement accuracy data is acquired by the accuracy check device 60 in time of execution of the accuracy check mode, and generated based on the determination result of the measurement accuracy transmitted to the main body 2 of the sphygmomanometer 1 through the communication line 70. The measurement accuracy data includes data indicating that the measurement accuracy satisfies a predetermined level set in advance and the blood pressure measurement can be executed, and data indicating that the measurement accuracy does not satisfy the predetermined level and the blood pressure measurement cannot be executed.

An air bladder (not shown) is arranged in the cuff 5, and the air bladder is pushed against the measurement site by wrapping the cuff 5 around the upper arm as the measurement site.

When executing the accuracy check mode, the accuracy check device 60 couples the connection plug 92 to the connector 6 of the main body 2 of the sphygmomanometer 1 so that an internal air system communicates to a measurement air system (both of which are not shown) incorporated in the main body 2. A CPU (Central Processing Unit) for controlling the entire sphygmomanometer 1 is communicable inside the main body 2 through the communication line 70. The communication line 70 may be wired or wireless.

The accuracy check device 60 includes an operation unit 64 such as a switch, and a display unit 62 for displaying the check result of the measurement accuracy.

The operation unit 64 includes a power switch 642 for instructing ON/OFF of the power supply, and a switch (hereinafter referred to as diagnosis term switch) 646 for instructing selection of the diagnosis item during the execution of the accuracy check mode. The diagnosis item includes a plurality of diagnosis items so that the operation performance can be diagnosed individually with respect to the components of the sphygmomanometer 1 with the measurement accuracy of the sphygmomanometer 1 as the base.

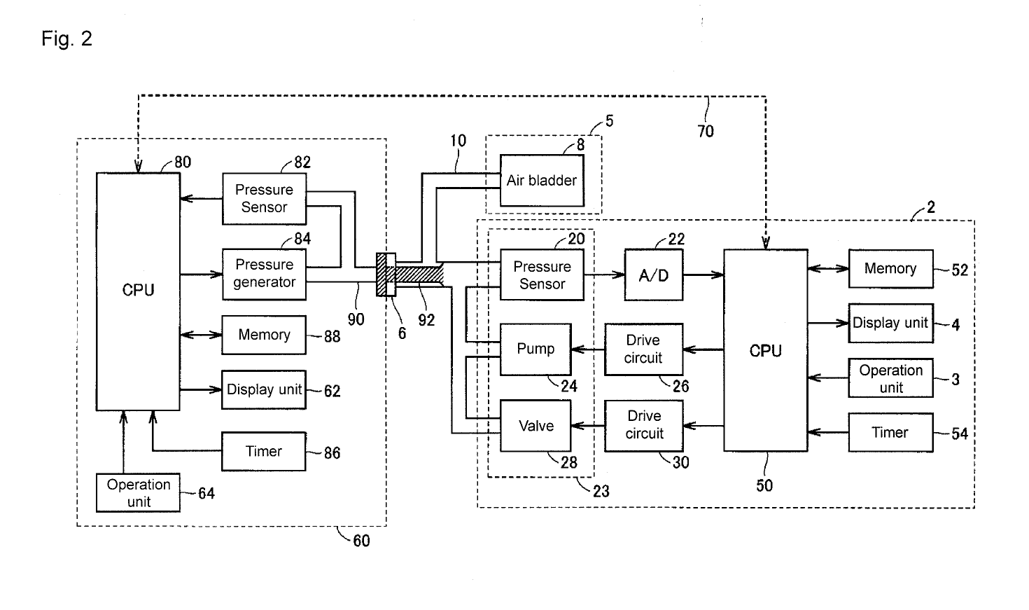

FIG. 2 is a block diagram showing a specific example of a hardware configuration of the sphygmomanometer 1 and the accuracy check device 60.

With reference to FIG. 2, the sphygmomanometer 1 includes the main body 2 and the cuff 5 to be wrapped around the upper arm as the measurement site, which are connected with each other by the air tube 10. The operation unit 3 such as a switch and the display unit 4 for displaying the measurement result are arranged on the front surface of the main body 2. An air bladder 8 is arranged in the cuff 5, and the air bladder 8 is pushed against the measurement site by wrapping the cuff 5 around the upper arm as the measurement site.

The air bladder 8 is connected to a measurement air system 23. The measurement air system 23 includes a pressure sensor 20 for measuring a change in internal pressure of the air bladder 8, a pump 24 for supplying/exhausting air with respect to the air bladder 8, and a valve 28.

The main body 2 of the sphygmomanometer 1 includes a CPU 50 for controlling the entire sphygmomanometer 1, an A/D (Analog to Digital) converter 22 connected to the measurement air system 23, a drive circuit 26 for driving the pump 24 and a drive circuit 30 for adjusting the opening and closing of the valve 28, a timer 54 for obtaining the measurement date and time, and a memory 52 for storing programs to be executed by the CPU 50 and measurement results.

The CPU 50 executes a predetermined program stored in the memory 52 based on an operation signal input from the operation unit 3, and outputs a control signal to the drive circuits 26, 30. The drive circuits 26, 30 drive the pump 24 and the valve 28 in response to the control signal to execute the blood pressure measurement operation.

The pressure sensor 20 detects the change in internal pressure of the air bladder 8, and inputs a detection signal to an amplifier (not shown). The input pressure signal is amplified to a predetermined amplitude by the amplifier, and input to the CPU 50 after being converted to a digital signal in the A/D converter 22. The CPU 50 executes a predetermined process based on the change in internal pressure of the air bladder 8 obtained from the pressure sensor 20, and outputs the control signal to the drive circuits 26, 30 according to the result. The CPU 50 also calculates a blood pressure value based on the change in internal pressure of the air bladder 8 obtained from the pressure sensor 20, and outputs the measurement result so as to be displayed by the display unit 4.

The opening and closing of the valve 28 is controlled by the drive circuit 30 according to the control signal from the CPU 50, and the valve 28 exhausts the air in the air bladder 8.

In the above configuration, the CPU 50 supplies power to each unit when the operation signal by the operation of the power switch 302 (FIG. 1) is input, and thereafter enters a standby state to wait for the input of the next operation signal. When receiving the input of the operation signal by the operation of the measurement switch 304 (FIG. 1), the “blood pressure measurement mode” is selected for the operation mode, and a series of blood pressure measurement operations is executed.

When receiving the input of the operation signal by the operation of the accuracy check mode switch 306 (FIG. 1) in the standby state, the “accuracy check mode” is selected for the operation mode, and a series of measurement accuracy check operations is executed.

The sphygmomanometer 1 further includes the connector 6 in the configuration for executing the measurement accuracy check operation. When performing the check operation of the measurement accuracy with respect to the sphygmomanometer 1, the connection plug 92 on the accuracy check device 60 side is coupled to the connector 6 so that the measurement air system 23 in the main body 2 communicates to the air system (air tube 90) of the accuracy check device 60.

Specifically, as shown in FIG. 2, the connection plug 92 has a cylindrical portion of a predetermined length, which cylindrical portion communicates the air tube 90 of the accuracy check device 60 to the measurement air system 23, and shields the same with respect to the air bladder 8.

The accuracy check device 60 includes a CPU 80 for controlling the entire accuracy check device 60, a pressure sensor 82 and a pressure generator 84 connected to the air tube 90, a display unit 62, a timer 86 for performing the timing operation and outputting timing data, an operation unit 64 such as a switch, and a memory 88 for storing programs to be executed by the CPU 80 and determination data of the measurement accuracy.

The CPU 80 executes a predetermined program stored in the memory 88 based on the operation signal input from the operation unit 64, and outputs the control signal to the pressure generator 84. The pressure generator 84 generates pressure based on a pressure generation pattern set in advance according to the control signal.

The pressure sensor 82 detects the change in internal pressure of the air tube 90, and inputs the detection signal to an amplifier (not shown). The input pressure signal is amplified to a predetermined amplitude by the amplifier, and input to the CPU 80 after being converted to a digital signal in an A/D converter (not shown). The CPU 80 executes a predetermined process based on the change in internal pressure of the air tube 90 obtained from the pressure sensor 82, and outputs the control signal to the pressure generator 84 according to the result. The CPU 80 also calculates a pressure (highest pressure) value based on the change in internal pressure of the air tube 90 obtained from the pressure sensor 82.

The CPU 80 also receives the blood pressure (systolic blood pressure) calculated by the CPU 50 of the sphygmomanometer 1 through the communication line 70. The CPU 80 then calculates the pressure deviation between the calculated highest pressure value and the systolic blood pressure value received from the CPU 50, and determines the measurement accuracy of the sphygmomanometer 1 based on the calculated pressure deviation. The determination result of the measurement accuracy is output to the display unit 62, and provided to the CPU 50 of the sphygmomanometer 1 through the communication line 70.

In the sphygmomanometer 1, the CPU 50 receives the determination result of the measurement accuracy through the communication line 70, and performs the process of storing data to the memory 52 with the date and time, when the measurement accuracy check operation is executed, obtained by the timer 54. The CPU 50 also determines whether or not the execution of the blood pressure measurement is possible based on the determination result of the measurement accuracy, and displays the determination result on the display unit 4.

(Functional Configuration)

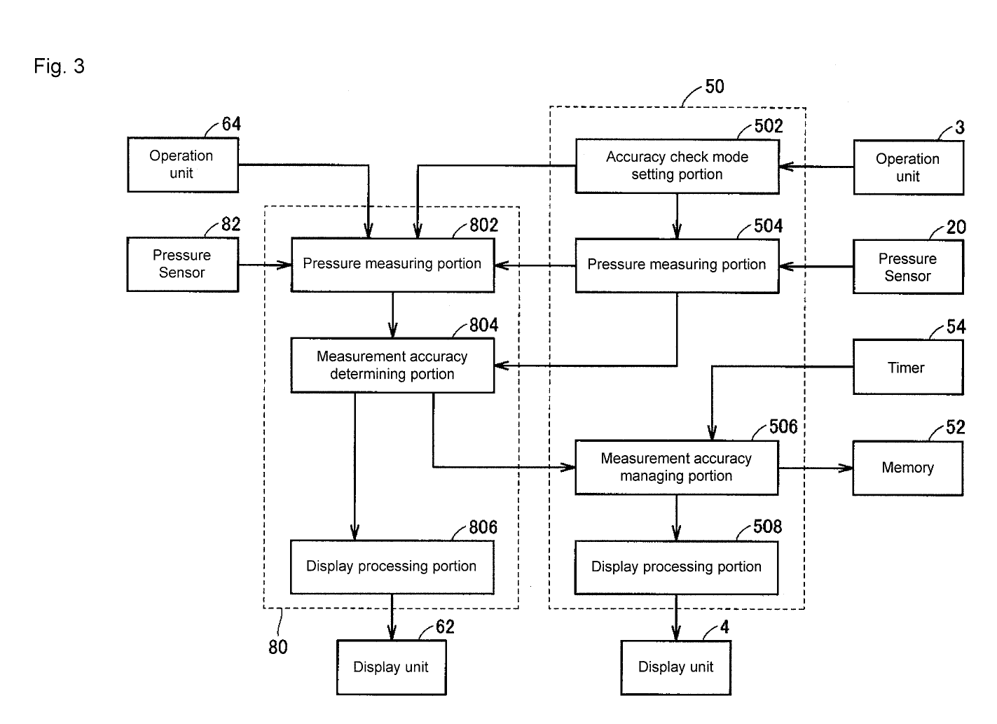

FIG. 3 is a block diagram showing a specific example of the functional configuration for performing the check operation of the measurement accuracy of the sphygmomanometer 1. The functions shown in FIG. 3 are functions executed by the CPU 50, 80 when the CPU 50, 80 executes a predetermined program stored in the memory 52, 88. Some or all of the functions shown in FIG. 3 may be implemented by hardware.

With reference to FIG. 3, the function for performing the measurement accuracy check operation of the sphygmomanometer 1 includes an accuracy check mode setting portion 502, a pressure measuring portion 504, a measurement accuracy managing portion 506, and a display processing portion 508 implemented by the CPU 50 of the sphygmomanometer 1, and a pressure measuring portion 802, a measurement accuracy determining portion 804, and a display processing portion 806 implemented by the CPU 80 of the accuracy check device 60. As described above, transmission and reception of data between the CPU 50 and the CPU 80 are carried out through the communication line 70 (FIG. 1).

The accuracy check mode setting portion 502 sets the operation mode of the sphygmomanometer 1 to the accuracy check mode in response to the input of the operation signal by the operation of the accuracy check mode switch 306 (FIG. 1). The accuracy check mode setting portion 502 generates an accuracy check request with respect to the pressure measuring portion 504, 802, and executes the measurement accuracy check operation of the sphygmomanometer 1.

Specifically, the pressure measuring portion 802 receives the operation signal by the operation of the diagnosis item switch 646 (FIG. 1) of the operation unit 64, and selects the diagnosis item from the plurality of diagnosis items set in advance. A case in which the measurement accuracy of the sphygmomanometer 1 is selected as the diagnosis item will be hereinafter described.

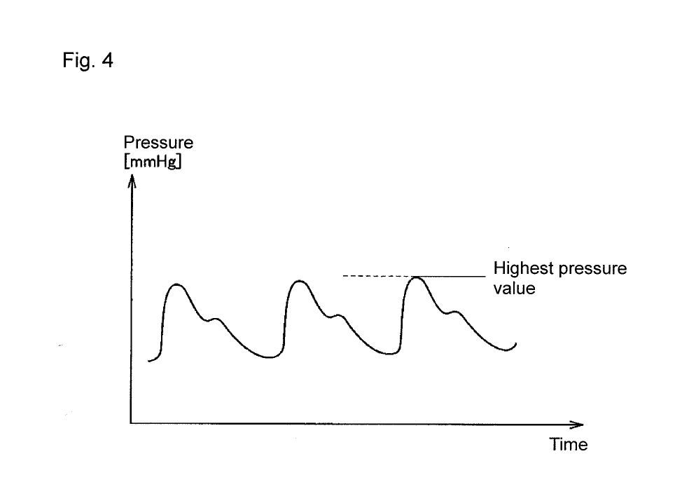

When diagnosing the measurement accuracy of the sphygmomanometer 1, the pressure measuring portion 802 controls the pressure generator 84 and generates a pseudo-pulse wave for expressing the change of the pulse pressure of the measuring site detected with the pressure sensor 20 of the sphygmomanometer 1 in time of normal blood pressure measurement in response to the measurement start request transmitted from the pressure measuring portion 504. FIG. 4 is a view for describing the pseudo-pulse wave generated by the pressure generator 84. With reference to FIG. 4, the pseudo-pulse wave shows the waveform in which the highest value of the pressure (highest pressure) is a predetermined value (e.g., 120 mmHg) set in advance.

In this case, the pressure measuring portion 802 detects the change in internal pressure of the air tube 90 with the pressure sensor 82, and calculates the highest pressure value based on the detected pressure. The calculated highest pressure value is output to the measurement accuracy determining portion 804.

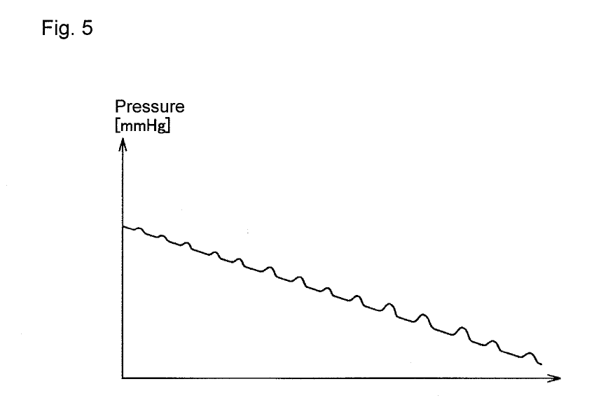

The pressure measuring portion 504 receives the operation signal by the operation of the measurement switch 304 (FIG. 1), generates the measurement start request and outputs the same to the pressure measuring portion 802, and executes the blood pressure measurement process. FIG. 5 is a view for describing the change in internal pressure of the air tube 90 detected with the pressure sensor 20. The pressure measuring portion 504 calculates the blood pressure (systolic blood pressure) value based on the change in internal pressure detected with the pressure sensor 20 shown in FIG. 5, and outputs the calculated systolic blood pressure value to the measurement accuracy determining portion 804.

When receiving the systolic blood pressure value from the pressure measuring portion 504 and also receiving the highest pressure value from the pressure measuring portion 802, the measurement accuracy determining portion 804 calculates the pressure deviation between the two pressure values. The measurement accuracy of the sphygmomanometer 1 is determined based on the calculated pressure deviation.

Specifically, the measurement accuracy determining portion 804 determines whether or not the calculated pressure deviation is smaller than or equal to a predetermined threshold value set in advance. If the calculated pressure deviation is smaller than or equal to the predetermined threshold value, the measurement accuracy determining portion 804 determines that the measurement accuracy of the sphygmomanometer 1 satisfies a predetermined level and is normal. If the calculated pressure deviation is greater than the predetermined threshold value, the measurement accuracy determining portion 804 determines that the measurement accuracy of the sphygmomanometer 1 does not satisfy the predetermined level and is abnormal. The measurement accuracy determining portion 804 outputs the determination result of the measurement accuracy to the display processing portion 806. The display processing portion 806 performs the process of displaying the determination result of the measurement accuracy on the display unit 62.

The measurement accuracy determining portion 804 further transmits the determination result of the measurement accuracy to the measurement accuracy managing portion 506 inside the CPU 50 through the communication line 70 (FIG. 1).

The measurement accuracy managing portion 506 receives the determination result of the measurement accuracy and performs the data storage process to the memory 52. In this case, the determination result of the measurement accuracy is stored in the memory 52 in association with the date and time, when the measurement accuracy check operation is executed, obtained by the timer 54.

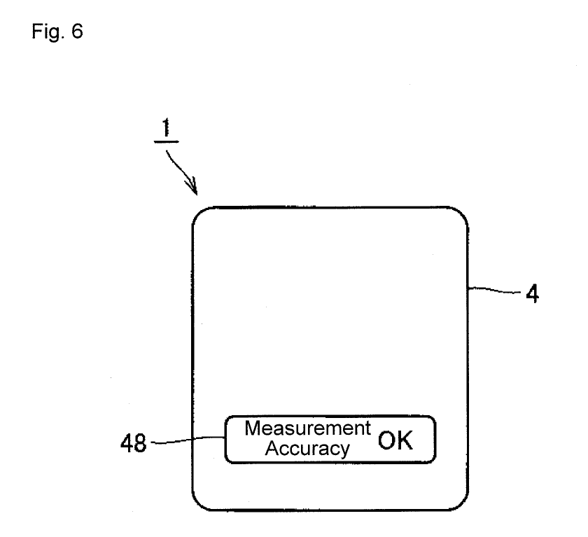

The measurement accuracy managing portion 506 determines whether or not the blood pressure measurement operation is executable based on the determination result of the measurement accuracy stored in the memory 52. The measurement accuracy managing portion 506 determines that the blood pressure measurement operation is executable if the measurement accuracy satisfies the predetermined level, and determination is made as normal. The display processing portion 508 performs the process of displaying the determination result on the display unit 4. FIG. 6 is a view showing a display example on the display unit 4. As shown in FIG. 6, when the measurement accuracy is high, this can be displayed in the display region 48 as a message such as “measurement accuracy OK”.

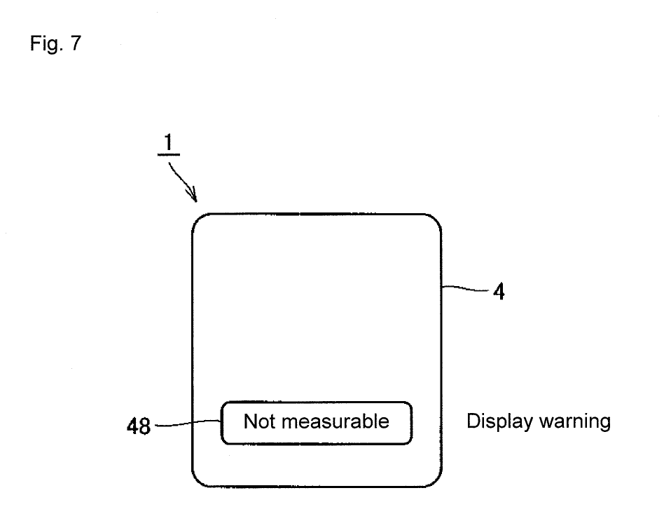

The measurement accuracy managing portion 506 determines that the blood pressure measurement operation is not executable if the measurement accuracy does not satisfy the predetermined level and determination is made as abnormal. The display processing portion 508 performs the process of displaying the determination result on the display unit 4. FIG. 7 is a view showing another display example on the display unit 4. As shown in FIG. 7, when the blood pressure measurement operation is not executable since the measurement accuracy is low, this can be displayed on the display unit 4 as a warning such as “not measurable”.

If determined that the blood pressure measurement operation is not executable, the process of prohibiting the execution of the blood pressure measurement operation of the next and subsequent times is performed in addition to a warning on the user.

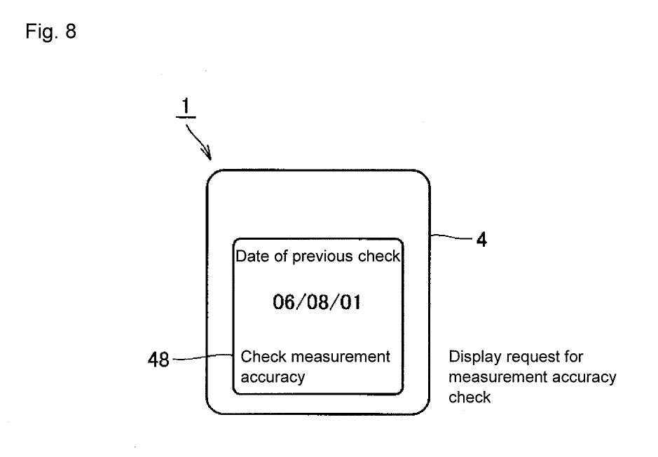

Furthermore, the measurement accuracy managing portion 506 determines whether or not the measurement accuracy check operation of the sphygmomanometer 1 needs to be performed based on the determination result of the measurement accuracy stored in the memory 52 and the timing information from the timer 54. Specifically, the measurement accuracy managing portion 506 times the elapsed time from the date and time at which the measurement accuracy check operation of the previous time is performed using the timer 54 so that the check of the measurement accuracy is performed at a predefined frequency, and determines whether or not the timed elapsed time exceeded a predetermined reference time. If the elapsed time exceeds a predetermined reference time, the measurement accuracy managing portion 506 determines that the measurement accuracy check operation is necessary. The display processing portion 508 performs the process of displaying the determination result on the display unit 4. FIG. 8 is a view showing another display example on the display unit 4. With reference to FIG. 8, if the measurement accuracy check operation is necessary, a display such as “check measurement accuracy” may be displayed with the date and time at which the measurement accuracy check operation of the previous time is performed as a notification urging the execution of the measurement accuracy check operation to the user.

The notification is not limited to the display of the display unit 4, and notification may be made to the user by lighting the Led (Light Emitting Diode), or by ringing a buzzer by means of an informing unit (not shown).

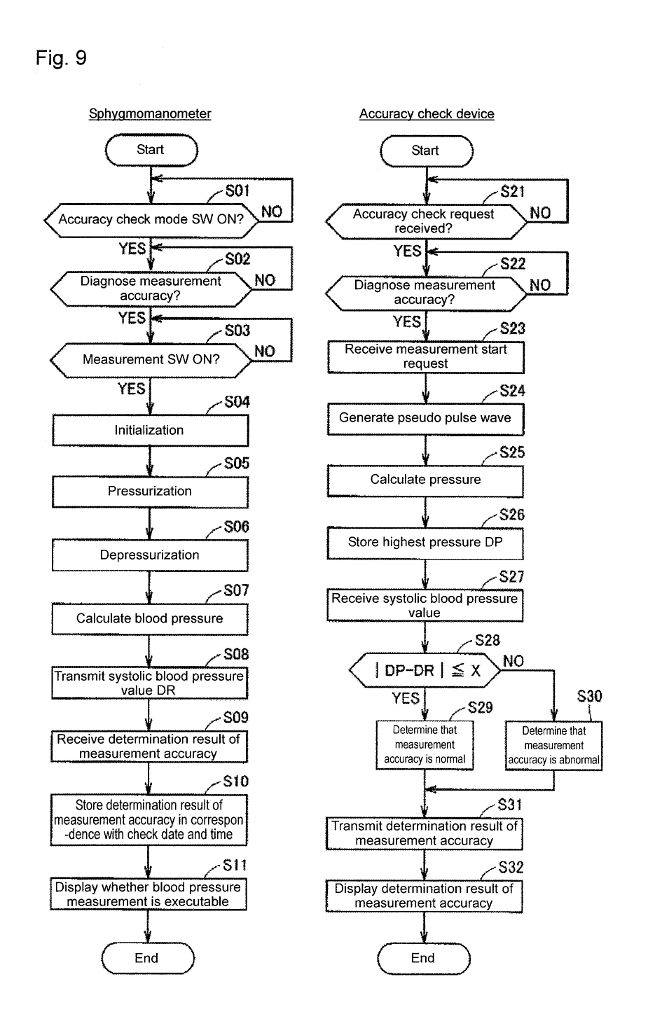

FIG. 9 is a flowchart for describing the measurement accuracy check operation of the sphygmomanometer 1 executed by the CPU 50 of the sphygmomanometer 1 and the CPU 80 of the accuracy check device 60. The flowchart of FIG. 9 is stored in the memories 52, 88 as a program in advance, and read and executed by the CPU 50, 80, respectively. The process shown in FIG. 9 is a process that starts when the power is supplied to the CPU 50, 80 after the power switch 302 of the sphygmomanometer 1 and the power switch 642 of the accuracy check device 60 are operated.

With reference to FIG. 9, on the sphygmomanometer 1 side, the CPU 50 first determines the presence of operation of the accuracy check mode switch 306 (FIG. 1) (step S01). If detected that the accuracy check mode switch 306 is operated (YES in step S01), the CPU 50 sets the operation mode of the sphygmomanometer 1 to the accuracy check mode. The CPU 50 also generates the accuracy check request, and transmits the same to the CPU 80 through the communication line 70 (FIG. 1).

Furthermore, when receiving the diagnosis item selected by the user from the CPU 80 of the accuracy check device 60, the CPU 50 determines whether or not the selected diagnosis item is the measurement accuracy of the sphygmomanometer 1 (step S02). If determined that the selected diagnosis item is the measurement accuracy of the sphygmomanometer 1 (YES in step S02), the CPU 50 executes the processes for blood pressure measurement shown in S03 to S07. Such processes are the same as the processes executed for the blood pressure measurement when the sphygmomanometer 1 is in the blood pressure measurement mode.

Specifically, the CPU 50 determines the presence of the operation of the measurement switch 304 (FIG. 1) (step S03). If detected that the measurement switch 304 is operated (YES in step S03), the CPU 50 controls each unit, and exhausts the air in the air tube 90 inside the accuracy device 60 and performs the 0 mmHg correction of the pressure sensor 20 as the initialization process of the sphygmomanometer 1 (step S04).

The CPU 50 then controls each unit and pressurizes the pressure in the air tube 90 up to about highest pressure (e.g., 120 mmHg)+40 mmHg of the pseudo-pulse wave (step S05). The pressure in the air tube 90 is then gradually depressurized (step S06). In the depressurization process, the pressure in the air tube 90 is detected with the pressure sensor 20, and the CPU 50 calculates the blood pressure (systolic blood pressure) value DR based on the relevant detected pressure (step S07). The calculated systolic blood pressure value DR is transmitted to the CPU 80 of the accuracy check device 60 through the communication line 70 (step S08). After transmitting the systolic blood pressure value DR, the CPU 50 is in the state waiting for the reception of the determination result of the measurement accuracy with respect thereto.

If the measurement accuracy determination result is received (step S09), the CPU 50 stores in the memory 52 the measurement accuracy determination result in association with the date and time, when the measurement accuracy check operation is executed, obtained by the timer 54 (step S10).

Furthermore, the CPU 50 determines whether or not the blood pressure measurement operation is executable based on the determination result of the measurement accuracy stored in the memory 52. The display as shown in FIG. 6 and FIG. 7 is made on the display unit 4 by such a determination result (step S11).

On the accuracy check device side 60, the state is the state waiting for the reception of the accuracy check request transmitted from the sphygmomanometer 1 (step S21). When receiving the accuracy check request from the sphygmomanometer 1 (YES in step S21), the CPU 80 determines whether or not the selected diagnosis item is the measurement accuracy of the sphygmomanometer 1 based on the operation signal input from the diagnosis item switch 646 (FIG. 1) (step S22). If determined that the selected diagnosis item is the measurement accuracy of the sphygmomanometer 1 (YES in step S22), the CPU 80 is in the state waiting for the reception of the measurement start request generated by the CPU 50 in cooperation with the operation of the measurement switch 304 (FIG. 1).

If the measurement start request is received (step S23), the CPU 80 controls each unit and generates the pseudo-pulse wave (step S24). In this case, the CPU 80 detects the pressure in the air tube 90 with the pressure sensor 82, calculates the pressure (highest pressure) value DP based on the detected pressure (step S25), and temporarily stores the same in the memory 52 (step S26). The CPU 80 is then in the state waiting for the reception of the systolic blood pressure value DR from the CPU 50 of the sphygmomanometer 1.

When the systolic blood pressure value DR is received from the CPU 50 (step S27), the CPU 80 calculates the pressure deviation (=|DP−DR|) between the systolic blood pressure value DR in the sphygmomanometer 1 and the highest pressure value DP in the accuracy check device 60, and determines whether or not the calculated pressure deviation is smaller than or equal to a predetermined threshold value X set in advance (step S28). If the calculated pressure deviation is smaller than or equal to the predetermined threshold value X (YES in step S28), the CPU 80 determines that the measurement accuracy of the sphygmomanometer 1 satisfies the predetermined level and is normal (step S29). If the calculated pressure deviation is greater than the predetermined threshold value X (NO in step S28), the CPU 80 determines that the measurement accuracy of the sphygmomanometer 1 does not satisfy the predetermined level and is abnormal (step S30). The CPU 80 transmits the determination result of the measurement accuracy to the CPU 50 of the sphygmomanometer 1 through the communication line 70 (step S31). Furthermore, the CPU 80 performs the process of displaying the determination result of the measurement accuracy on the display unit 62 (step S32).

With the above configuration, the user can check the measurement accuracy of the sphygmomanometer 1 by causing the sphygmomanometer 1 to execute the normal blood pressure measurement operation with the sphygmomanometer 1 connected to the accuracy check device 60. Two pressure sensors thus do not need to be installed in the main body 2 of the sphygmomanometer 1, whereby the measurement accuracy of the sphygmomanometer 1 can be checked with a simple and inexpensive device configuration.

If the measurement accuracy of the sphygmomanometer 1 does not satisfy the predetermined level and determination is made as abnormal, notification is made to the user that the blood pressure measurement operation is disabled or the blood pressure measurement operation is forcibly prohibited so that the continuation of the blood pressure measurement operation with the measurement accuracy in the abnormal state can be avoided.

Furthermore, with the configuration of making a notification to urge the execution of the measurement accuracy check operation to the user so that the measurement accuracy is periodically checked, the measurement accuracy of the sphygmomanometer 1 can be maintained in a high state. As a result, reliability on the measurement accuracy can be enhanced and effectiveness of the daily blood pressure management can be enhanced.

According to the measurement accuracy check system of the sphygmomanometer 1 of the present embodiment, the operation performance can be individually diagnosed with respect to the components of the sphygmomanometer 1 in addition to the measurement accuracy check operation. Therefore, the causes of lowering of the measurement accuracy can be specified by further diagnosing the operation performance for each component of the sphygmomanometer 1 when the measurement accuracy of the sphygmomanometer 1 is determined as abnormal.

Specifically, when diagnosing the operation performance on the components of the sphygmomanometer 1, the diagnosis item is first selected in accordance with the operation of the diagnosis item switch 646 (FIG. 1) of the accuracy check device 60 by the user. The diagnosis item includes the airtightness of the measurement air system 23, the dynamic characteristics etc. of the pressure sensor 20, the pump 24, and the valve 28, and the like.

When the diagnosis item is selected, the measurement air system 23 is controlled so as to generate pressure in the air tube 10 of the sphygmomanometer 1 with the pressure generation pattern optimum for diagnosing the operation performance of the components to be diagnosed. The pressure in the air tube 10 in this case is detected by the pressure sensor 20, and the operation performance of each component is diagnosed based on the detected pressure.

(Diagnosis of Airtightness of Measurement Air System)

First, the operation for diagnosing the airtightness of the measurement air system 23 of the sphygmomanometer 1 will be described.

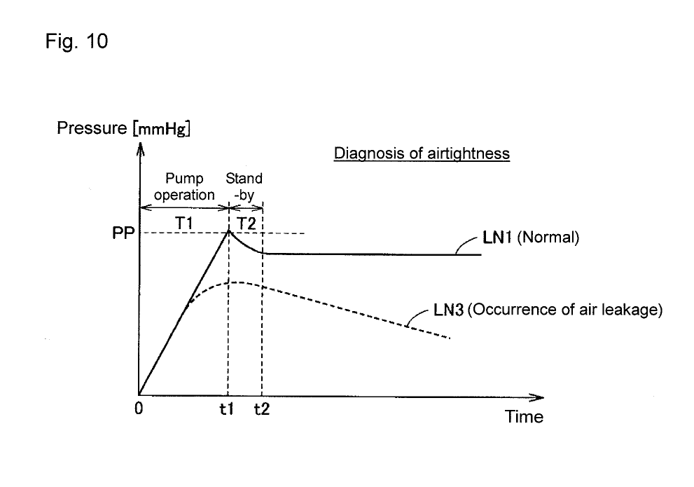

FIG. 10 is a view showing a pressure generation pattern when diagnosing the airtightness of the measurement air system 23.

With reference to FIG. 10, when airtightness of the measurement air system 23 is selected for the diagnosis item, the CPU 50 controls the drive circuit 26 and operates the pump 24 for a predetermined period T1 to pressurize the interior of the air tube 10 in the sphygmomanometer 1. The predetermined period T1 is set in advance to a time necessary for the pump 24 to pressurize the pressure in the air tube 10 to a predetermined pressure reference value PP.

After elapse of the predetermined period T1, the CPU 50 stops the operation of the measurement air system 23 for a predetermined period T2 to be in the standby state. This is so that an accurate pressure value is detected after the pressure in the air tube 10 stabilizes by providing a standby state as the pressure in the air tube 10 is unstable immediately after pressurization and an accurate pressure cannot be detected.

At time t2, at which the predetermined period T2 has elapsed, the CPU 50 detects the pressure in the air tube 10 with the pressure sensor 20, and transmits the detected pressure value PR to the CPU 80 through the communication line 70. The CPU 80 calculates a pressure deviation between the detected pressure value PR and the predetermined pressure reference value PP, and determines whether or not the pressure deviation is lower than or equal to a predetermined threshold value.

If the airtightness of the measurement air system 23 is normal, the pressure in the air tube 10 shows a substantially constant value after time t2, as shown with a line LN1 in FIG. 10. If the airtightness of the measurement air system 23 is abnormal (i.e., air leakage occurred), the pressure in the air tube 10 is greatly lower than the predetermined pressure reference value PP and continues to decrease after time t2, as shown with a line LN3 in FIG. 10.

Therefore, the CPU 80 determines that the airtightness of the measurement air system 23 is normal when the pressure deviation between the detected pressure value PR and the predetermined pressure reference value PP is lower than or equal to a predetermined threshold value. If the pressure deviation between the detected pressure value PR and the predetermined pressure reference value PP is greater than the predetermined threshold value, determination is made that the airtightness of the measurement air system 23 is abnormal. The CPU 80 then displays the determination result on the display unit 62. As shown in FIG. 1, the display unit 62 displays the determination result for every diagnosis item along with the determination result of the measurement accuracy of the sphygmomanometer 1.

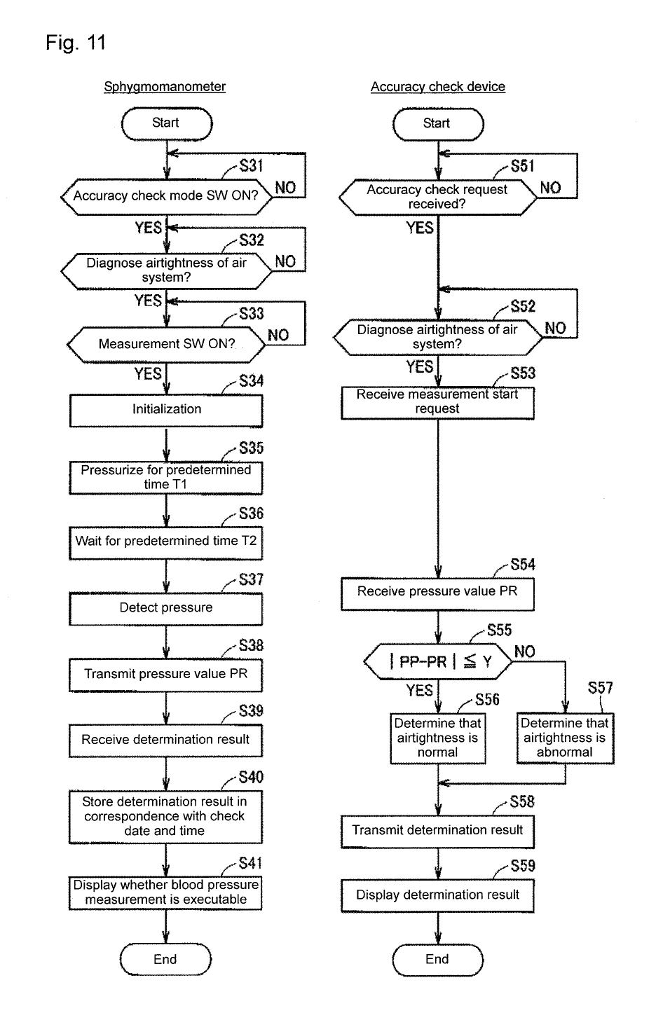

FIG. 11 is a flowchart for describing the diagnosis operation of the airtightness of the measurement air system 23 in the sphygmomanometer 1 executed by the CPU 50 of the sphygmomanometer 1 and the CPU 80 of the accuracy check device 60. The flowchart of FIG. 11 is stored in the memories 52, 88 as a program in advance, and read and executed by the CPU 50, 80, respectively. The process shown in FIG. 11 is a process that starts when the power is supplied to the CPU 50, 80 after the power switch 302 of the sphygmomanometer 1 and the power switch 642 of the accuracy check device 60 are operated.

With reference to FIG. 11, on the sphygmomanometer 1 side, the CPU 50 first determines the presence of operation of the accuracy check mode switch 306 (FIG. 1) (step S31). If detected that the accuracy check mode switch 306 is operated (YES in step S31), the CPU 50 sets the operation mode of the sphygmomanometer 1 to the accuracy check mode. The CPU 50 also generates the accuracy check request, and transmits the same to the CPU 80.

Furthermore, when receiving the diagnosis item selected by the user from the CPU 80 of the accuracy check device 60, the CPU 50 determines whether or not the selected diagnosis item is the airtightness of the measurement air system 23 (step S32). If determined that the selected diagnosis item is the airtightness of the measurement air system 23 (YES in step S32), the CPU 50 generates pressure in the air tube 10 according to the predetermined pressure generation pattern (FIG. 10), and detects the pressure change at the time with the pressure sensor 20.

Specifically, the CPU 50 determines the presence of the operation of the measurement switch 304 (FIG. 1) (step S33). If detected that the measurement switch 304 is operated (YES in step S33), the CPU 50 controls each unit, and exhausts the air in the air tube 90 inside the accuracy device 60 and performs the 0 mmHg correction of the pressure sensor 20 as the initialization process of the sphygmomanometer 1 (step S34).

The CPU 50 then controls each unit and pressurizes the pressure in the air tube 10 for a predetermined period T1 (step S35). The pressure in the air tube 10 is then pressurized up to about a predetermined pressure reference value PP. After having the measurement air system 23 in the standby state for the predetermined period T2 (step S36), the CPU 50 detects the pressure in the air tube 10 with the pressure sensor 20 (step S37). The CPU 50 transmits the detected pressure value PR to the CPU 80 of the accuracy check device 60 through the communication line 70 (step S38). After transmitting the pressure value PR, the CPU 50 is in the state waiting for the reception of the airtightness determination result with respect thereto.

If the airtightness determination result of the measurement air system 23 is received (step S39), the CPU 50 stores in the memory 52 the determination result in association with the date and time, when the diagnosis operation of the airtightness of the measurement air system 23 is executed, obtained by the timer 54 (step S40).

Furthermore, the CPU 50 determines whether or not the blood pressure measurement operation is executable based on the determination result of the measurement accuracy stored in the memory 52. The display as shown in FIG. 6 and FIG. 7 is made on the display unit 4 by such a determination result (step S41).

On the accuracy check device side 60, the state is the state waiting for the reception of the accuracy check request transmitted from the sphygmomanometer 1 (step S51). When receiving the accuracy check request from the sphygmomanometer 1 (YES in step S51), the CPU 80 determines whether or not the selected diagnosis item is the airtightness of the measurement air system 23 based on the operation signal input from the diagnosis item switch 646 (FIG. 1) (step S52). If determined that the selected diagnosis item is the airtightness of the measurement air system 23 (YES in step S52), the CPU 80 is in the state waiting for the reception of the measurement start request generated by the CPU 50 in cooperation with the operation of the measurement switch 304 (FIG. 1).

If the measurement start request is received (step S53), the CPU 80 is in the state waiting for the reception of the pressure detection value PR from the CPU 50 of the sphygmomanometer 1.

If the pressure detection value PR is received from the CPU 50 (step S54), the CPU 80 calculates the pressure deviation (=|PP−PR|) between the pressure detection value PR and the predetermined pressure reference value PP, and determines whether or not the calculated pressure deviation is smaller than or equal to a predetermined threshold value Y set in advance (step S55). If the calculated pressure deviation is smaller than or equal to the predetermined threshold value Y, the CPU 80 determines that the airtightness of the measurement air system 23 is normal (step S56). If the calculated pressure deviation is greater than the predetermined threshold value Y, the CPU 80 determines that the airtightness of the measurement air system 23 is abnormal (step S57). The CPU 80 transmits the determination results to the CPU 50 of the sphygmomanometer 1 through the communication line 70 (step S58). Furthermore, the CPU 80 performs the process of displaying the determination result on the display unit 62 (step S59).

With the execution of the flowchart of FIG. 11, the dynamic characteristics of the pressure sensor 20 can be diagnosed in addition to diagnosing the airtightness of the measurement air system 23.

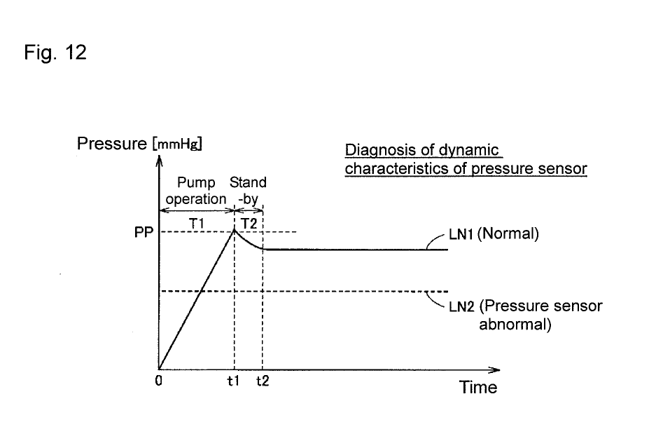

FIG. 12 is a view showing a pressure generation pattern when diagnosing the dynamic characteristics of the pressure sensor 20. The pressure generation pattern of FIG. 12 is the same as the pressure generation pattern of FIG. 10.

In other words, the CPU 50 operates the pump 24 for the predetermined period T1 to pressurize the interior of the air tube 10 to the predetermined pressure reference value PP, and then enters the standby state for the predetermined period T2. After elapse of the predetermined period T2, the CPU 50 detects the pressure in the air tube 10 with the pressure sensor 20, and transmits the detected pressure value to the CPU 80.

If the dynamic characteristics of the pressure sensor 20 are normal, the pressure in the air tube 10 shows a substantially constant value after time t2, as shown with a line LN1 in FIG. 12. If the dynamic characteristics of the pressure sensor 20 are abnormal, the pressure in the air tube 10 shows a value greatly deviated from the predetermined pressure reference value PP, as shown with a line LN2 in FIG. 12.

Therefore, the CPU 80 determines that the dynamic characteristics of the pressure sensor 20 are normal when the pressure deviation between the detected pressure value PR and the predetermined pressure reference value PP is lower than or equal to a predetermined threshold value. The CPU 80 determines that the dynamic characteristics of the pressure sensor 20 are abnormal when the pressure deviation between the detected pressure value PR and the predetermined pressure reference value PP is greater than the predetermined threshold value.

(Diagnosis of Dynamic Characteristics of Pump)

The operation of diagnosing the dynamic characteristics of the pump 24 of the sphygmomanometer 1 will be described below.

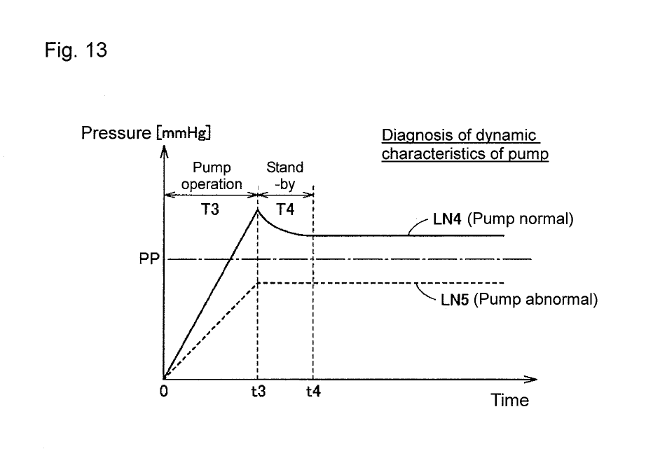

FIG. 13 is a view showing a pressure generation pattern when diagnosing the dynamic characteristics of the pump 24.

With reference to FIG. 13, if the dynamic characteristics of the pump 24 are selected for the diagnosis item, the CPU 50 controls the drive circuit 26 and operates the pump 24 for a predetermined period T3 at a maximum ability to pressurize the interior of the air tube 10 in the sphygmomanometer 1. The predetermined period T3 is set in advance to a time sufficient for the pump 24 to pressurize the pressure in the air tube 10 to greater than or equal to a predetermined pressure reference value PP.

After elapse of the predetermined period T3, the CPU 50 stops the operation of the measurement air system 23 for a predetermined period T4 to be in the standby state. At time t4, at which the predetermined period T4 has elapsed, the CPU 50 detects the pressure in the air tube 10 with the pressure sensor 20, and transmits the detected pressure value PR to the CPU 80 through the communication line 70. The CPU 80 compares the detected pressure value PR and a predetermined pressure reference value PP.

In this case, if the dynamic characteristics of the pump 24 are normal, the pressure in the air tube 10 is greater than or equal to a predetermined pressure reference value PP, as shown with a line LN4 in FIG. 13. If the dynamic characteristics of the pump 24 are abnormal, the pressure in the air tube 10 is lower than a predetermined pressure reference value PP, as shown with a line LN5 in FIG. 13.

Therefore, the CPU 80 determines that the dynamic characteristics of the pump 24 are normal if the detected pressure value PR is greater than or equal to a predetermined reference value PP. The CPU 80 determines that the dynamic characteristics of the pump 24 are abnormal if the detected pressure value PR is lower than the predetermined reference value PP. The CPU 80 displays the determination result on the display unit 62. As shown in FIG. 1, the display unit 62 displays the determination result on the dynamic characteristics of the pump 24 with the determination result of the measurement accuracy of the sphygmomanometer 1.

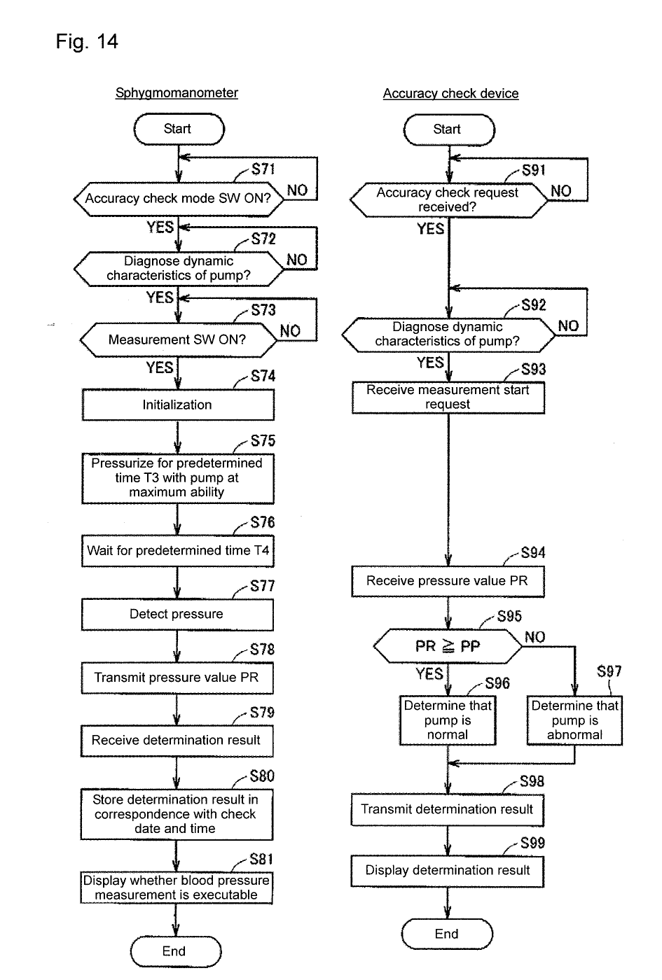

FIG. 14 is a flowchart for describing the diagnosis operation of the dynamic characteristics of the pump 24 in the sphygmomanometer 1 executed by the CPU 50 of the sphygmomanometer 1 and the CPU 80 of the accuracy check device 60. The flowchart of FIG. 14 is stored in the memories 52, 88 as a program in advance, and read and executed by the CPU 50, 80, respectively. The process shown in FIG. 14 is a process that starts when the power is supplied to the CPU 50, 80 after the power switch 302 of the sphygmomanometer 1 and the power switch 642 of the accuracy check device 60 are operated.

With reference to FIG. 14, on the sphygmomanometer 1 side, the CPU 50 first determines the presence of operation of the accuracy check mode switch 306 (FIG. 1) (step S71). If detected that the accuracy check mode switch 306 is operated (YES in step S71), the CPU 50 sets the operation mode of the sphygmomanometer 1 to the accuracy check mode. The CPU 50 also generates the accuracy check request, and transmits the same to the CPU 80.

Furthermore, when receiving the diagnosis item selected by the user from the CPU 80 of the accuracy check device 60, the CPU 50 determines whether or not the selected diagnosis item is the dynamic characteristics of the pump 24 (step S72). If determined that the selected diagnosis item is the dynamic characteristics of the pump 24 (YES in step S72), the CPU 50 generates pressure in the air tube 10 according to the predetermined pressure generation pattern (FIG. 13), and detects the pressure change at the time with the pressure sensor 20.

Specifically, the CPU 50 determines the presence of the operation of the measurement switch 304 (FIG. 1) (step S73). If detected that the measurement switch 304 is operated (YES in step S73), the CPU 50 controls each unit, and exhausts the air in the air tube 90 inside the accuracy device 60 and performs the 0 mmHg correction of the pressure sensor 20 as the initialization process of the sphygmomanometer 1 (step S74).

The CPU 50 then controls each unit and drives the pump 24 at a maximum ability to pressurize the pressure in the air tube 10 for the predetermined period T3 (step S75). The pressure in the air tube 10 is then pressurized to greater than or predetermined pressure reference value PP. After having the measurement air system 23 in the standby state for the predetermined period T4 (step S76), the CPU 50 detects the pressure in the air tube 10 with the pressure sensor 20 (step S77). The CPU 50 transmits the detected pressure value PR to the CPU 80 of the accuracy check device 60 through the communication line 70 (step S78). After transmitting the pressure value PR, the CPU 50 is in the state waiting for the reception of the dynamic characteristics determination result of the pump 24 with respect thereto.

If the determination result of the dynamic characteristics of the pump 24 is received (step S79), the CPU 50 stores in the memory 52 the determination result in association with the date and time, when the diagnosis operation of the dynamic characteristics of the pump 24 is executed, obtained by the timer 54 (step S80).

Furthermore, the CPU 50 determines whether or not the blood pressure measurement operation is executable based on the determination result stored in the memory 52. The display as shown in FIG. 6 and FIG. 7 is made on the display unit 4 by such a determination result (step S81).

On the accuracy check device side 60, the state is the state waiting for the reception of the accuracy check request transmitted from the sphygmomanometer 1 (step S91). When receiving the accuracy check request from the sphygmomanometer 1 (YES in step S91), the CPU 80 determines whether or not the selected diagnosis item is the dynamic characteristics of the pump 24 based on the operation signal input from the diagnosis item switch 646 (FIG. 1) (step S92). If determined that the selected diagnosis item is the dynamic characteristics of the pump 24 (YES in step S92), the CPU 80 is in the state waiting for the reception of the measurement start request generated by the CPU 50 in cooperation with the operation of the measurement switch 304 (FIG. 1).

If the measurement start request is received (step S93), the CPU 80 is in the state waiting for the reception of the pressure detection value PR from the CPU 50 of the sphygmomanometer 1.

If the pressure detection value PR is received from the CPU 50 (step S94), the CPU 80 determines whether or not the pressure detection value PR is greater than or equal to a predetermined pressure reference value PP (step S95). If the pressure detection value PR is greater than or equal to the predetermined pressure reference value PP, the CPU 80 determines that the dynamic characteristics of the pump 24 are normal (step S96). If the pressure detection value PR is smaller than the predetermined pressure reference value PP, the CPU 80 determines that the dynamic characteristics of the pump 24 are abnormal (step S97). The CPU 80 transmits the determination results to the CPU 50 of the sphygmomanometer 1 through the communication line 70 (step S98). Furthermore, the CPU 80 performs the process of displaying the determination result on the display unit 62 (step S99).

(Diagnosis of Dynamic Characteristics of Valve)

Lastly, the operation of diagnosing the dynamic characteristics of the valve 28 of the sphygmomanometer 1 will be described.

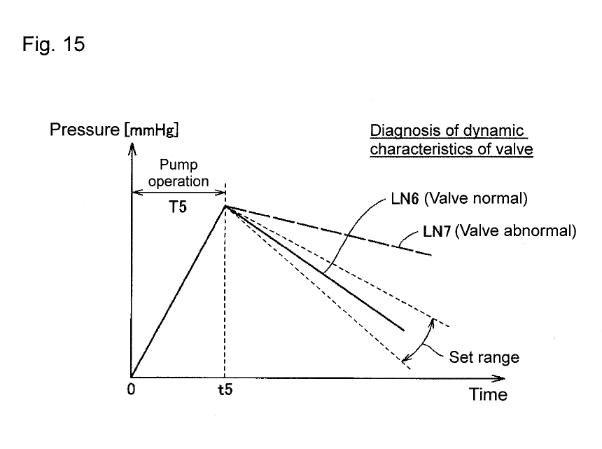

FIG. 15 is a view showing a pressure generation pattern when diagnosing the dynamic characteristics of the valve 28.

With reference to FIG. 15, if the dynamic characteristics of the valve 28 are selected for the diagnosis item, the CPU 50 controls the drive circuit 26 and operates the pump 24 for a predetermined period T5 to pressurize the interior of the air tube 10 in the sphygmomanometer 1. At time t5, at which the predetermined period T5 has elapsed, the CPU 50 opens the valve 28 to open the measurement air system 23 to atmosphere thereby starting depressurization. The CPU 50 detects the pressure in the air tube 10 after time t5 with the pressure sensor 20, and calculates the reduction speed (hereinafter depressurization speed) Vp of the pressure value based on the detected pressure value and the timing information from the timer 54. The CPU 50 transmits the calculated depressurization speed Vp to the CPU 80 through the communication line 70. The CPU 80 determines whether or not the calculated depressurization speed Vp is within a range of the depressurization speed set in advance.

In this case, if the dynamic characteristics of the valve 28 are normal, the pressure in the air tube 10 reduces at the depression speed within a predetermined set range, as shown with a line LN6 in FIG. 15. If the dynamic characteristics of the valve 28 are abnormal, the pressure in the air tube 10 reduces at the depression speed outside predetermined set range, as shown with a line LN7 in FIG. 15.

Therefore, the CPU 80 determines that the dynamic characteristics of the valve 28 are normal if the calculated depressurization speed Vp is within the predetermined set range. The CPU 80 determines that the dynamic characteristics of the valve 28 are abnormal if the calculated depressurization speed Vp is outside the predetermined set range. The CPU 80 displays the determination result on the display unit 62. As shown in FIG. 1, the display unit 62 displays the determination result on the dynamic characteristics of the valve 28 with the determination result of the measurement accuracy of the sphygmomanometer 1.

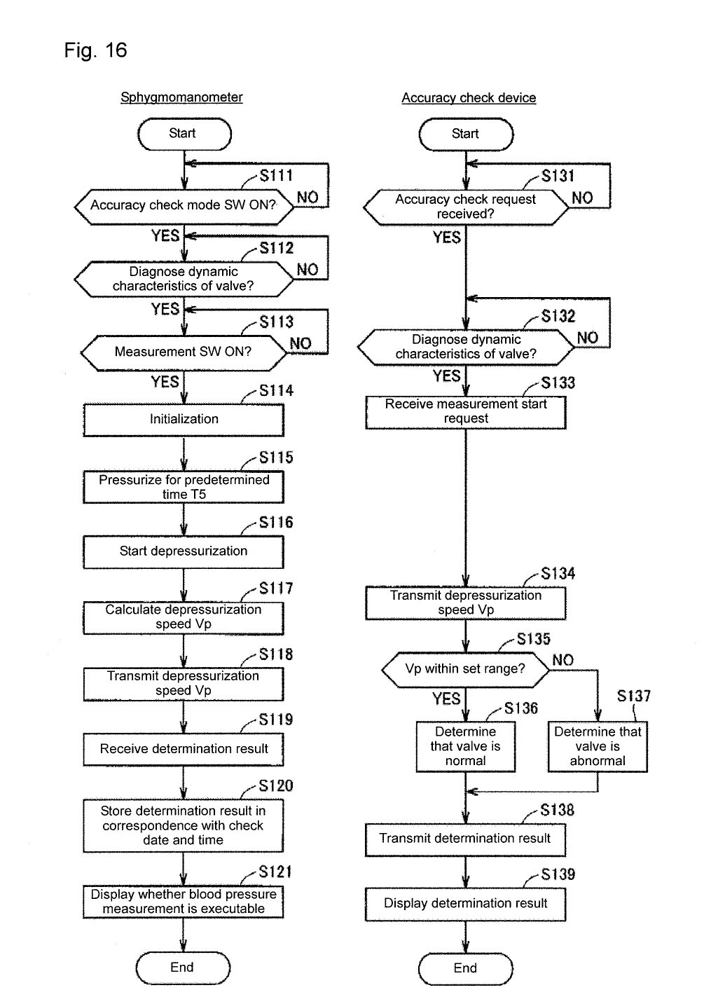

FIG. 16 is a flowchart for describing the diagnosis operation of the dynamic characteristics of the valve 28 in the sphygmomanometer 1 executed by the CPU 50 of the sphygmomanometer 1 and the CPU 80 of the accuracy check device 60. The flowchart of FIG. 16 is stored in the memories 52, 88 as a program in advance, and read and executed by the CPU 50, 80, respectively. The process shown in FIG. 16 is a process that starts when the power is supplied to the CPU 50, 80 after the power switch 302 of the sphygmomanometer 1 and the power switch 642 of the accuracy check device 60 are operated.

With reference to FIG. 16, on the sphygmomanometer 1 side, the CPU 50 first determines the presence of operation of the accuracy check mode switch 306 (FIG. 1) (step S111). If detected that the accuracy check mode switch 306 is operated (YES in step S111), the CPU 50 sets the operation mode of the sphygmomanometer 1 to the accuracy check mode. The CPU 50 also generates the accuracy check request, and transmits the same to the CPU 80.

Furthermore, when receiving the diagnosis item selected by the user from the CPU 80 of the accuracy check device 60, the CPU 50 determines whether or not the selected diagnosis item is the dynamic characteristics of the valve 28 (step S112). If determined that the selected diagnosis item is the dynamic characteristics of the valve 28 (YES in step S112), the CPU 50 starts depressurization after pressurizing the interior of the air tube 10 according to the predetermined pressure generation pattern (FIG. 15), and detects the pressure change at the time with the pressure sensor 20.

Specifically, the CPU 50 determines the presence of the operation of the measurement switch 304 (FIG. 1) (step S113). If detected that the measurement switch 304 is operated (YES in step S113), the CPU 50 controls each unit, and exhausts the air in the air tube 90 inside the accuracy device 60 and performs the 0 mmHg correction of the pressure sensor 20 as the initialization process of the sphygmomanometer 1 (step S114).

The CPU 50 then controls each unit to pressurize the pressure in the air tube 10 for the predetermined period T5 (step S115). The CPU 50 then opens the valve 28 to start depressurization (step S116), and detects the pressure in the air tube 10 with the pressure sensor 20. The CPU 50 calculates the depressurization speed Bp based on the detected pressure value and the timing information from the timer 54 (step S117), and transmits the calculated depressurization speed Vp to the accuracy check device 60 through the communication line 70 (step S118). After transmitting the depressurization speed Vp, the CPU 50 is in the state waiting for the determination result of the dynamic characteristics of the valve 28 with respect thereto.

If the determination result of the dynamic characteristics of the valve 28 is received (step S119), the CPU 50 stores in the memory 52 the determination result in association with the date and time, when the diagnosis operation of the dynamic characteristics of the valve 28 is executed, obtained by the timer 54 (step S120).

Furthermore, the CPU 50 determines whether or not the blood pressure measurement operation is executable based on the determination result stored in the memory 52. The display as shown in FIG. 6 and FIG. 7 is made on the display unit 4 by such a determination result (step S121).

On the accuracy check device side 60, the state is the state waiting for the reception of the accuracy check request transmitted from the sphygmomanometer 1 (step S131). When receiving the accuracy check request from the sphygmomanometer 1 (YES in step S131), the CPU 80 determines whether or not the selected diagnosis item is the dynamic characteristics of the valve 28 based on the operation signal input from the diagnosis item switch 646 (FIG. 1) (step S132). If determined that the selected diagnosis item is the dynamic characteristics of the valve 28 (YES in step S132), the CPU 80 is in the state waiting for the reception of the measurement start request generated by the CPU 50 in cooperation with the operation of the measurement switch 304 (FIG. 1).

If the measurement start request is received (step S133), the CPU 80 is in the state waiting for the reception of the depressurization speed Vp from the CPU 50 of the sphygmomanometer 1.

If the depressurization speed Vp is received from the CPU 50 (step S134), the CPU 80 determines whether or not the depressurization speed Vp is within a predetermined set range (step S135). If the depressurization speed Vp is within the predetermined set range, the CPU 80 determines that the dynamic characteristics of the valve 28 are normal (step S136). If the depressurization speed Vp is outside the predetermined set range, the CPU 80 determines that the dynamic characteristics of the valve 28 are abnormal (step S137). The CPU 80 transmits the determination results to the CPU 50 of the sphygmomanometer 1 through the communication line 70 (step S138). Furthermore, the CPU 80 performs the process of displaying the determination result on the display unit 62 (step S139).

[Variant]

In the first embodiment, the accuracy check device 60 is configured to determine the measurement accuracy of the sphygmomanometer 1, but the measurement accuracy may be determined on the sphygmomanometer 1 side.

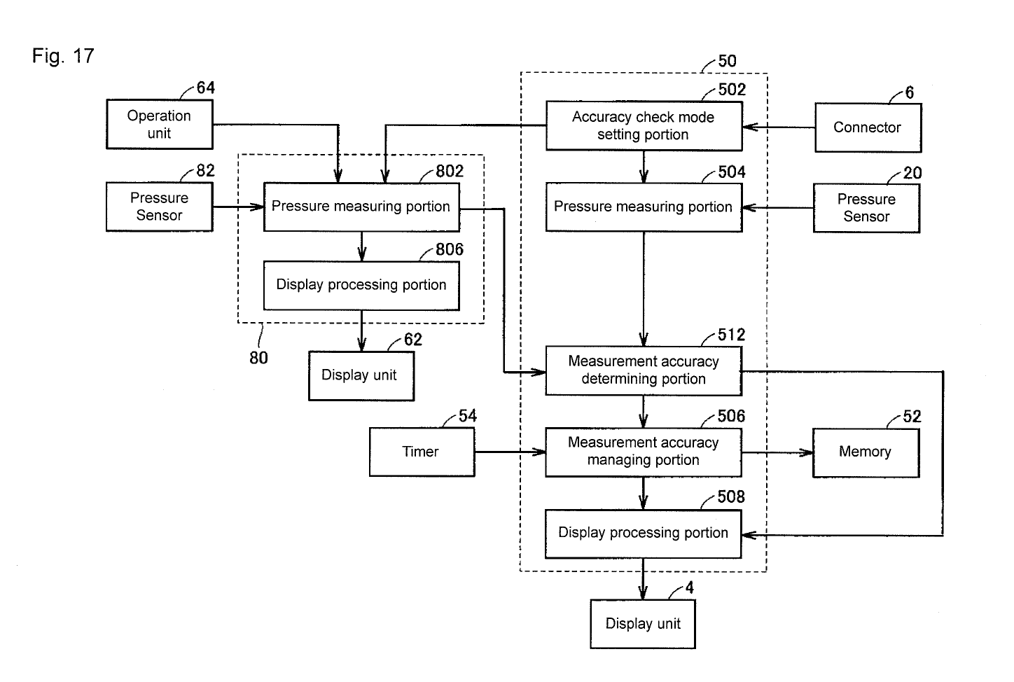

FIG. 17 is a block diagram showing a specific example of the functional configuration for performing the check operation of the measurement accuracy of the sphygmomanometer 1 in the measurement accuracy check system of the sphygmomanometer according to a variant of the first embodiment. The functions shown in FIG. 17 are functions executed by the CPU 50, 80 when the CPU 50, 80 executes a predetermined program stored in the memory 52, 88, respectively. Some or all of the functions shown in FIG. 17 may be implemented by hardware.

With reference to FIG. 17, the function for performing the measurement accuracy check operation of the sphygmomanometer 1 includes the accuracy check mode setting portion 502, the pressure measuring portion 504, a measurement accuracy determining portion 512, the measurement accuracy managing portion 506, and the display processing portion 508 implemented by the CPU 50 of the sphygmomanometer 1, and the pressure measuring portion 802 and the display processing portion 806 implemented by the CPU 80 of the accuracy check device 60.

The functional configuration shown in FIG. 17 is obtained by replacing the measurement accuracy determining portion 804 implemented by the CPU 80 in the functional configurations shown in FIG. 3 with the measurement accuracy determining portion 512 implemented by the CPU 50. Therefore, the detailed description on the sites same as those in the configuration of FIG. 3 will not be repeated.

In the present variant, the connector 6 (FIG. 1) includes a coupling detection sensor (not shown) for detecting the coupling state of the connector 6 and the connection plug 92. The accuracy check mode setting portion 502 implemented by the CPU 50 detects that the measurement accuracy check operation is executable by a coupling signal from the coupling detection sensor, and sets the operation mode of the sphygmomanometer 1 to the accuracy check mode. The accuracy check mode setting portion 502 then generates the accuracy check request with respect to the pressure measuring portions 504, 802, and executes the measurement accuracy check operation of the sphygmomanometer 1.

Specifically, the pressure measuring portion 802 generates a pseudo-pulse wave for expressing the change of the pulse pressure of the measuring site detected with the pressure sensor 20 of the sphygmomanometer 1 in time of normal blood pressure measurement in response to the measurement start request transmitted from the pressure measuring portion 504. The pressure measuring portion 802 then detects the change in internal pressure of the air tube 90 with the pressure sensor 82, and calculates the highest pressure value based on the detected pressure. The calculated highest pressure value is transmitted to the measurement accuracy determining portion 512 through the communication line 70.

The pressure measuring portion 504 receives the operation signal by the operation of the measurement switch 304 (FIG. 1), and executes the blood pressure measurement process. The pressure measuring portion 504 calculates the blood pressure (systolic blood pressure) value based on the change in internal pressure detected with the pressure sensor 20, and outputs the calculated systolic blood pressure value to the measurement accuracy determining portion 512.

When receiving the systolic blood pressure value from the pressure measuring portion 504 and also receiving the highest pressure value from the pressure measuring portion 802, the measurement accuracy determining portion 512 calculates the pressure deviation between the two pressure values. The measurement accuracy of the sphygmomanometer 1 is determined based on the calculated pressure deviation. The determination method of the detailed measurement accuracy is the same as the determination method of the measurement accuracy determining portion 804. The measurement accuracy determining portion 512 outputs the determination result of the measurement accuracy to the measurement accuracy managing portion 506 and the display processing portion 508.

The measurement accuracy managing portion 506 receives the determination result of the measurement accuracy and performs the data storage process to the memory 52 and also determines whether or not the blood pressure measurement operation is executable by the above-described method. The display processing portion 508 performs the process of displaying the determination result on the display unit 4, and performs the process of displaying the determination result regarding whether or not the blood pressure measurement operation is executable on the display unit 4. Furthermore, if the measurement accuracy managing portion 506 determines that the measurement accuracy check operation of the sphygmomanometer 1 is necessary based on the determination result of the measurement accuracy stored in the memory 52 and the timing information from the timer 54, the display processing portion 508 performs a process of displaying on the display unit 4 a notification urging the execution of the measurement accuracy check operation to the user.

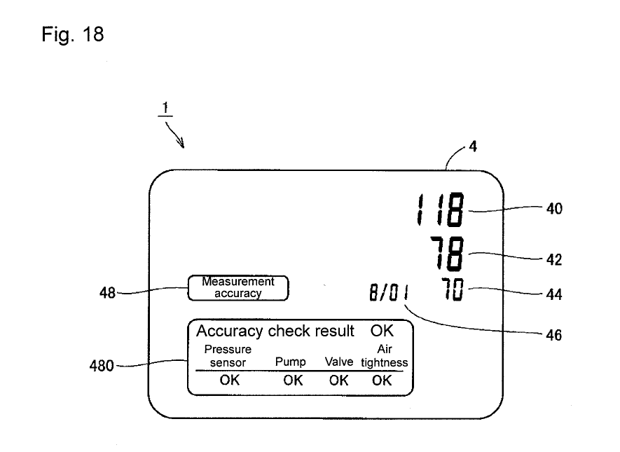

FIG. 18 is a view showing a display example on the display unit 4. As shown in FIG. 18, the display unit 4 includes the display region 48 for displaying measurement accuracy data indicating the measurement accuracy of the sphygmomanometer 1 at the date and time of the blood pressure measurement, and the display region 480 showing the determination result obtained by the measurement accuracy check operation.

With the above configuration, the user can check the measurement accuracy of the sphygmomanometer 1 by causing the sphygmomanometer 1 to execute the normal blood pressure measurement operation with the sphygmomanometer 1 connected to the accuracy check device 60. As a result, two pressure sensors do not need to be installed in the main body 2 of the sphygmomanometer 1, whereby the measurement accuracy of the sphygmomanometer 1 can be checked with a simple and inexpensive device configuration.

The check operation of the measurement accuracy is to be periodically performed to maintain the measurement accuracy of the sphygmomanometer 1, where the user does not need to own the accuracy check device 60 individually so that the cost can be further lowered by commonly using the accuracy check device 60 among a plurality of sphygmomanometers.

Moreover, if determined that the measurement accuracy of the sphygmomanometer 1 does not satisfy the predetermined level, notification is made to the user that the blood pressure measurement operation is disabled or the blood pressure measurement operation is forcibly prohibited so that the continuation of the blood pressure measurement operation with the measurement accuracy in the abnormal state can be avoided. The measurement accuracy of the sphygmomanometer 1 can be maintained in a high state with the configuration of making a notification to urge the execution of the measurement accuracy check operation to the user so that the measurement accuracy is periodically checked. As a result, reliability on the measurement accuracy can be enhanced and effectiveness of the daily blood pressure management can be enhanced.

### Second Embodiment

In a second embodiment, a measurement accuracy check system of a sphygmomanometer in which the check function of the measurement accuracy is given to the sphygmomanometer 1 side without performing the communication between the sphygmomanometer 1 and the accuracy check device 60 will be described. The hardware configuration of the measurement accuracy check system of the sphygmomanometer of the second embodiment is basically similar to the configuration of the measurement accuracy check system of the sphygmomanometer of the first embodiment, and differs in that the communication line 70 is not arranged and in that the check operation of the measurement accuracy is performed on the sphygmomanometer 1 side, as hereinafter described.

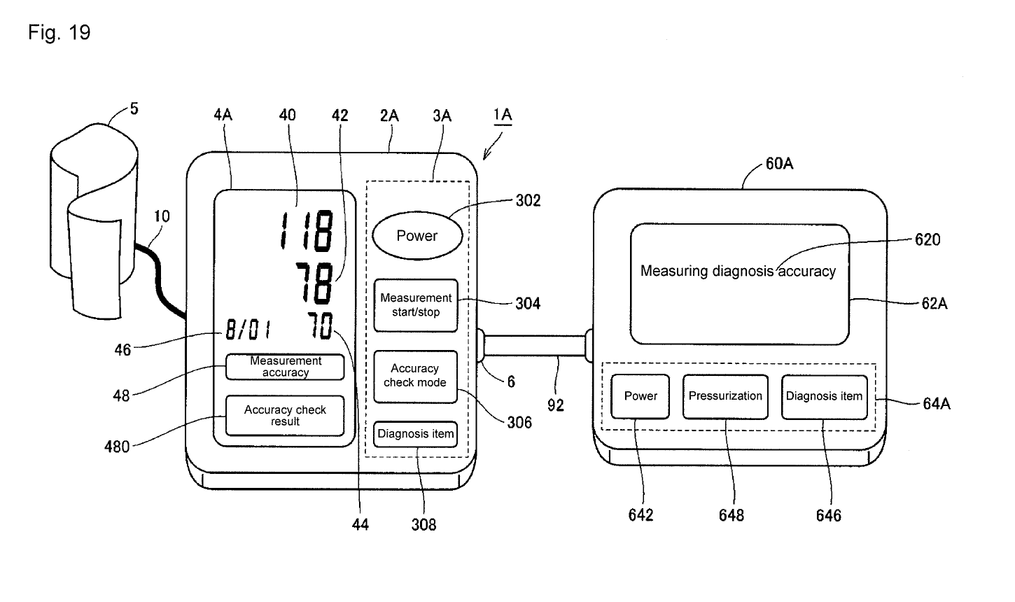

FIG. 19 is a schematic view of an outer appearance of the measurement accuracy check system of the sphygmomanometer according to the second embodiment of the present invention.

With reference to FIG. 19, the measurement accuracy check system of the sphygmomanometer according to the second embodiment includes a sphygmomanometer 1A, an accuracy check device 60A, and the connection plug 92.

The connection plug 92 is coupled to the connector 6 arranged in the main body 2A of the sphygmomanometer 1 when performing the check operation of the measurement accuracy on the sphygmomanometer 1A.

The sphygmomanometer 1A includes a main body 2A and a cuff 5 to be wrapped around the upper arm as the measurement site, which are connected with each other by an air tube 10. An operation unit 3A such as a switch and a display unit 4A for displaying the measurement result are arranged on the front surface of the main body 2A.

The operation unit 3A includes the power switch 302 for instructing ON/OFF of the power supply, the measurement switch 304 for instructing start/stop of the measurement, the accuracy check mode switch 306, and the diagnosis item switch 308 for instructing selection of the diagnosis item.

The display unit 4 includes display regions 40 to 46 for displaying the measurement result. The display regions 40 to 44 show systolic blood pressure data indicating the systolic blood pressure, diastolic blood pressure data indicating the diastolic blood pressure, and number of pulse data indicating the number of pulses. The display region 46 displays time data indicating the date and time of the blood pressure measurement.

The display unit 4A also includes the display region 48 for displaying measurement accuracy data indicating the measurement accuracy of the sphygmomanometer 1 at the date and time of the blood pressure measurement, and the display region 480 for displaying the check result of the measurement accuracy acquired during the execution of the accuracy check mode.

When executing the accuracy check mode, the accuracy check device 60A couples the connection plug 92 to the connector 6 of the main body 2A of the sphygmomanometer 1A, so that an internal air system communicates to a measurement air system (both of which are not shown) incorporated in the main body 2A. The accuracy check device 60A includes an operation unit 64A such as a switch, and a display unit 62A for displaying the check result of the measurement accuracy.

The operation unit 64 includes the power switch 642 for instructing ON/OFF of the power supply, a pressurization switch 648 for instructing start of pressurization, and a diagnosis item switch 308 for instructing selection of the diagnosis item.

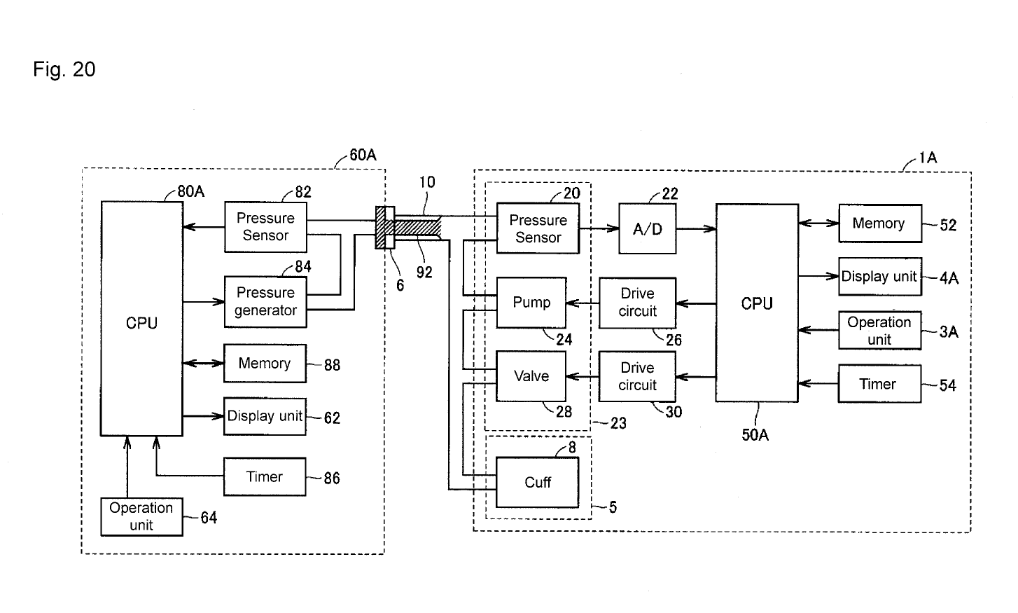

FIG. 20 is a block diagram showing a specific example of the hardware configuration of the sphygmomanometer 1A and the accuracy check device 60A.

With reference to FIG. 20, the sphygmomanometer 1A includes the main body 2A and the cuff 5 to be wrapped around the upper arm as the measurement site, which are connected with each other by an air tube 10. The operation unit 3A such as a switch and the display unit 4A for displaying the measurement result are arranged on the front surface of the main body 2A. An air bladder 8 is arranged in the cuff 5, and the air bladder 8 is pushed against the measurement site by wrapping the cuff 5 around the upper arm as the measurement site.

The air bladder 8 is connected to the measurement air system 23. The measurement air system 23 includes the pressure sensor 20 for measuring the change in internal pressure of the air bladder 8, the pump 24 for supplying/exhausting air with respect to the air bladder 8, and the valve 28.

The main body 2A of the sphygmomanometer 1A includes a CPU 50A for controlling the entire sphygmomanometer 1A, the A/D converter 22 connected to the measurement air system 23, the drive circuit 26 for driving the pump 24 and the drive circuit 30 for adjusting the opening and closing of the valve 28, the timer 54 for obtaining the measurement date and time, and the memory 52 for storing programs to be executed by the CPU 50A and measurement results.

When the operation signal by the operation of the power switch 302 (FIG. 19) is input, the CPU 50A supplies power to each unit, and then waits for the input of the next operation signal. When receiving the input of the operation signal by the operation of the measurement switch 304 (FIG. 19), the “blood pressure measurement mode” is selected for the operation mode, and a series of blood pressure measurement operations is executed.

When receiving the input of the operation signal by the operation of the accuracy check mode switch 306 (FIG. 19) in the standby state, the “accuracy check mode” is selected for the operation mode, and a series of measurement accuracy check operations is executed.

The sphygmomanometer 1A further includes the connector 6 as a configuration for executing the measurement accuracy check operation. When performing the check operation of the measurement accuracy on the sphygmomanometer 1A, the connection plug 92 on the accuracy check device 60A side is coupled to the connector 6 so that the measurement air system 23 in the main body 2A communicates to the air system (air tube 90) of the accuracy check device 60A.

As described above, the connection plug 92 communicates the air tube 90 of the accuracy check device 60A to the measurement air system 23, and shields the same with respect to the air bladder 8.

The accuracy check device 60 includes a CPU 80A for controlling the entire accuracy check device 60A, the pressure sensor 82 and the pressure generator 84 connected to the air tube 90, a display unit 62A, the timer 86 for performing the timing operation and outputting timing data, an operation unit 64A such as a switch, and the memory 88 for storing programs to be executed by the CPU 80A.

The CPU 80A executes a predetermined program stored in the memory 88 based on the operation signal input from the operation unit 64A, and outputs the control signal to the pressure generator 84. The pressure generator 84 generates pressure based on a pressure generation pattern set in advance according to the control signal.

The pressure sensor 82 detects the change in internal pressure of the air tube 90, and inputs the detection signal to an amplifier (not shown). The input pressure signal is amplified to a predetermined amplitude by the amplifier, and input to the CPU 80A after being converted to a digital signal in an A/D converter (not shown). The CPU 80A executes a predetermined process based on the change in internal pressure of the air tube 90 obtained from the pressure sensor 82, and outputs the control signal to the pressure generator 84 according to the result.

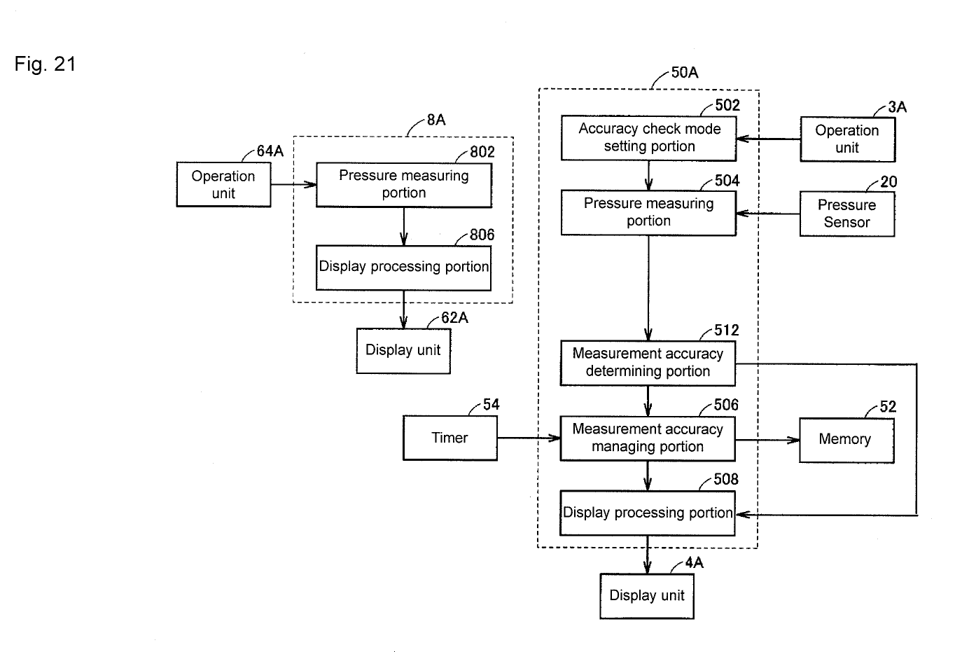

FIG. 21 is a block diagram showing a specific example of the function configuration for performing the check operation of the measurement accuracy of the sphygmomanometer 1A. The functions shown in FIG. 21 are functions executed by the CPU 50A, 80A when the CPU 50A, 80A executes a predetermined program stored in the memory 52, 88. Some or all of the functions shown in FIG. 21 may be implemented by hardware.

With reference to FIG. 21, the function for performing the measurement accuracy check operation of the sphygmomanometer 1A includes the accuracy check mode setting portion 502, the pressure measuring portion 504, the measurement accuracy determining portion 512, the measurement accuracy managing portion 506, and the display processing portion 508 implemented by the CPU 50A of the sphygmomanometer 1A, and the pressure measuring portion 802 and the display processing portion 806 implemented by the CPU 80A of the accuracy check device 60A.

The accuracy check mode setting portion 502 sets the operation mode of the sphygmomanometer 1 to the accuracy check mode in response to the input of the operation signal by the operation of the accuracy check mode switch 306 (FIG. 19). The accuracy check mode setting portion 502 then generates the accuracy check request with respect to the pressure measuring portions 504, 802, and executes the measurement accuracy check operation of the sphygmomanometer 1.

The setting of the accuracy check mode in the sphygmomanometer 1 may be carried out by detecting that the measurement accuracy check operation is in the executable state by a coupling signal from the coupling detection sensor, installed in the connector 6, for detecting the coupling state of the connector 6 and the connection plug 92 other than being carried out in response to the operation signal from the accuracy check mode switch 306. Alternatively, the setting may be carried out by detecting the pressure signal output from the air tube 90 of the accuracy check device 60A to the air tube 10 of the sphygmomanometer 1A.

The pressure measuring portion 802 receives the operation signal by the operation of the pressurization switch 648 (FIG. 19) of the operation unit 64A, and generates the pseudo-pulse wave for expressing the change of the pulse pressure of the measuring site detected with the pressure sensor 20 of the sphygmomanometer 1A in time of normal blood pressure measurement. The pseudo-pulse wave has a waveform as shown in FIG. 4, and is set in advance so that the highest value of the pressure (highest pressure value) becomes a predetermined value (e.g., 120 mmHg).

The pressure measuring portion 504 receives the operation signal by the operation of the measurement switch 304 (FIG. 1), and executes the blood pressure measurement process. The pressure measuring portion 504 calculates the blood pressure (systolic blood pressure) value based on the pressure detected with the pressure sensor 20, and outputs the calculated systolic blood pressure value to the measurement accuracy determining portion 512.

When receiving the systolic blood pressure value from the pressure measuring portion 504, the measurement accuracy determining portion 512 calculates the pressure deviation between the systolic blood pressure value and the systolic blood pressure value (e.g., 120 mmHg) of the pseudo-pulse wave set in advance. The measurement accuracy of the sphygmomanometer 1A is determined based on the calculated pressure deviation.

Specifically, the measurement accuracy determining portion 512 determines whether or not the calculated pressure deviation difference is smaller than or equal to a predetermined threshold value set in advance. If the calculated pressure deviation is smaller than or equal to the predetermined threshold value, the measurement accuracy determining portion 512 determines that the measurement accuracy of the sphygmomanometer 1A satisfies a predetermined level and is normal. If the calculated pressure deviation is greater than the predetermined threshold value, the measurement accuracy determining portion 512 determines that the measurement accuracy of the sphygmomanometer 1A does not satisfy a predetermined level and is abnormal. The measurement accuracy determining portion 512 outputs the determination result of the measurement accuracy to the display processing portion 508. The display processing portion 508 performs a process of displaying the determination result of the measurement accuracy on the display unit 4A.

The measurement accuracy determining portion 512 also outputs the determination result of the measurement accuracy to the measurement accuracy managing portion 506. The measurement accuracy managing portion 506 receives the determination result of the measurement accuracy. and performs the data storage process to the memory 52. The determination result of the measurement accuracy is stored in the memory 52 in association with the date and time, when the measurement accuracy check operation is executed, obtained by the timer 54.

The measurement accuracy managing portion 506 determines whether or not the blood pressure measurement operation is executable based on the determination result of the measurement accuracy stored in the memory 52. The measurement accuracy managing portion 506 determines that the blood pressure measurement operation is executable if the measurement accuracy satisfies the predetermined level, and determination is made as normal. The display processing portion 508 performs the process of displaying the determination result on the display unit 4. The measurement accuracy managing portion 506 determines that the blood pressure measurement operation is not executable if the measurement accuracy does not satisfy the predetermined level, and determination is made as abnormal. The display processing portion 508 performs the process of displaying the determination result on the display unit 4.

If determined that the blood pressure measurement operation is not executable, the process of prohibiting the execution of the blood pressure measurement operation of the next and subsequent times is performed in addition to a warning on the user.

Furthermore, the measurement accuracy managing portion 506 determines whether or not the measurement accuracy check operation of the sphygmomanometer 1 needs to be performed based on the determination result of the measurement accuracy stored in the memory 52 and the timing information from the timer 54. Specifically, the measurement accuracy managing portion 506 times the elapsed time from the date and time at which the measurement accuracy check operation of the previous time is performed using the timer 54 so that the check of the measurement accuracy is performed at a predefined frequency, and determines whether or not the timed elapsed time exceeded a predetermined reference time. If the elapsed time exceeds a predetermined reference time, the measurement accuracy managing portion 506 determines that the measurement accuracy check operation is necessary. The display processing portion 508 performs the process of displaying the determination result on the display unit 4 as a notification urging execution of the measurement accuracy check operation to the user.

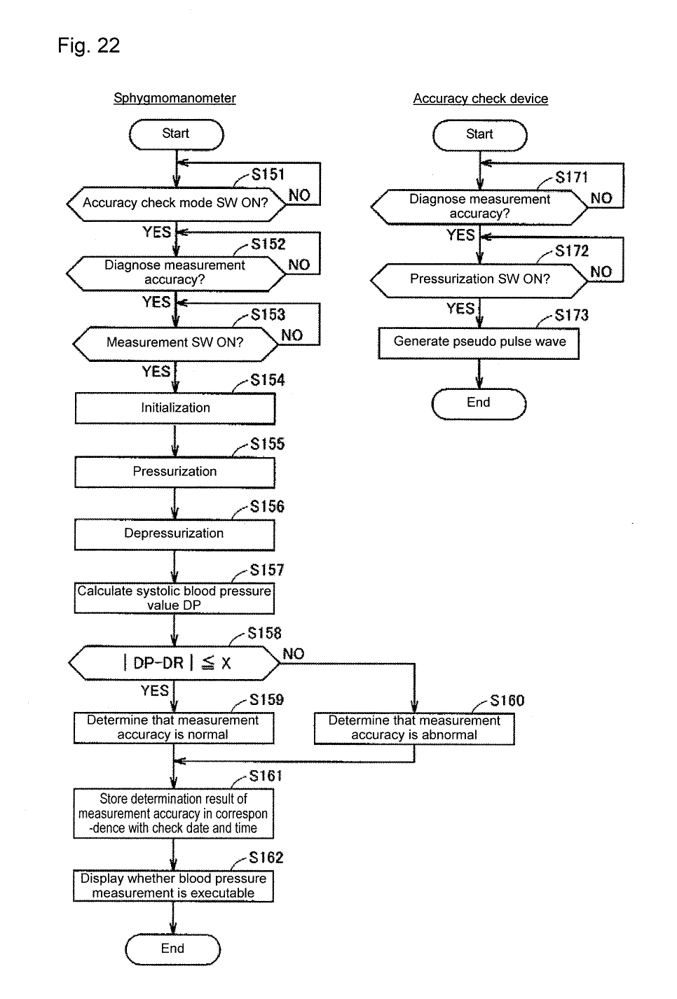

FIG. 22 is a flowchart for describing the measurement accuracy check operation of the sphygmomanometer 1A executed by the CPU 50A of the sphygmomanometer 1A and the CPU 80A of the accuracy check device 60A. The flowchart of FIG. 22 is stored in the memories 52, 88 as a program in advance, and read and executed by the CPU 50A, 80A, respectively. The process shown in FIG. 22 is a process that starts when the power is supplied to the CPU 50A, 80A after the power switch 302 of the sphygmomanometer 1A and the power switch 642 of the accuracy check device 60A are operated.

With reference to FIG. 22, on the sphygmomanometer 1A side, the CPU 50A first determines the presence of operation of the accuracy check mode switch 306 (FIG. 19) (step S151). If detected that the accuracy check mode switch 306 is operated (YES in step S151), the CPU 50A sets the operation mode of the sphygmomanometer 1A to the accuracy check mode.

Furthermore, the CPU 50A determines whether or not the selected diagnosis item is the measurement accuracy of the sphygmomanometer 1A (step S152) based on the operation signal by the operation of the diagnosis item switch 308 (FIG. 19). If determined that the selected diagnosis item is the measurement accuracy of the sphygmomanometer 1 (YES in step S152), the CPU 50A executes the processes for blood pressure measurement shown in S153 to S157. Such processes are the same as the processes executed for the blood pressure measurement when the sphygmomanometer 1A is in the blood pressure measurement mode.

Specifically, the CPU 50A determines the presence of the operation of the measurement switch 304 (FIG. 19) (step S153). If detected that the measurement switch 304 is operated (YES in step S153), the CPU 50A controls each unit, and exhausts the air in the air tube 90 inside the accuracy device 60 and performs the 0 mmHg correction of the pressure sensor 20 as the initialization process of the sphygmomanometer 1A (step S154).

The CPU 50A then controls each unit and pressurizes the pressure in the air tube 90 up to about highest pressure+40 mmHg of the pseudo-pulse wave (step S155). The pressure in the air tube 90 is then gradually depressurized (step S156). In the depressurization process, the pressure in the air tube 90 is detected with the pressure sensor 20, and the CPU 50A calculates the blood pressure (systolic blood pressure) value DR based on the relevant detected pressure (step S157).

The CPU 50A calculates the pressure deviation (=|DP−DR|) between the calculated systolic blood pressure value DR and the systolic blood pressure value DP of the pseudo-pulse wave set in advance, and determines whether or not the calculated pressure deviation is smaller than or equal to a predetermined threshold value X set in advance (step S158). If the calculated pressure deviation is smaller than or equal to the predetermined threshold value X, the CPU 50A determines that the measurement accuracy of the sphygmomanometer 1A satisfies the predetermined level and is normal (step S159). If the calculated pressure deviation is greater than the predetermined threshold value X, the CPU 50A determines that the measurement accuracy of the sphygmomanometer 1A does not satisfy the predetermined level and is abnormal (step S160). The CPU 50A performs the process of displaying the determination result of the measurement accuracy on the display unit 4a, and also stores the same in the memory in association with the date and time, when the measurement accuracy check operation is executed, obtained by the timer (step S161).

Furthermore, the CPU 50A determines whether or not the blood pressure measurement operation is executable based on the determination result of the measurement accuracy stored in the memory 52, and displays the determination result on the display unit 4A in the mode shown in FIG. 6 and FIG. 7 (step S162).

On the accuracy check device side 60A, the CPU 80A first determines whether or not the selected diagnosis item is the measurement accuracy of the sphygmomanometer 1 based on the operation signal input from the diagnosis item switch 646 (FIG. 19) (step S171). If determined that the selected diagnosis item is the measurement accuracy of the sphygmomanometer 1A (YES in step S171), the CPU 80A determines the presence of the operation of the pressurization switch 648 (FIG. 19) (step S172). If the operation of the pressurization switch 648 is detected (YES in step S172), the CPU 80A controls each unit and generates the pseudo-pulse wave for a predetermined period (step S173). The predetermined period is set in advance to include the time necessary for the blood pressure measurement process in the sphygmomanometer 1A.

With the above configuration, the user can check the measurement accuracy of the sphygmomanometer 1A by causing the sphygmomanometer 1A to execute the normal blood pressure measurement operation with the sphygmomanometer 1 connected to the accuracy check device 60. As a result, the measurement accuracy can be checked with a simple and inexpensive device configuration.

Furthermore, if the measurement accuracy of the sphygmomanometer 1A does not satisfy the predetermined level, notification is made to the user that the blood pressure measurement operation is disabled or the blood pressure measurement operation is forcibly prohibited so that the continuation of the blood pressure measurement operation with the measurement accuracy in the abnormal state can be avoided. The measurement accuracy of the sphygmomanometer 1A can be maintained in a high state by making a notification urging the execution of the measurement accuracy check operation to the user so that the measurement accuracy is periodically checked.

As a result, reliability on the measurement accuracy can be enhanced and effectiveness of the daily blood pressure management can be enhanced.

In the present embodiment as well, the diagnosis of the operation performance can be performed individually on the components of the sphygmomanometer 1A in addition to the check operation of the measurement accuracy. When diagnosing the operation performance of the components of the sphygmomanometer 1A, the measurement air system 23 is controlled to generate pressure in the air tube 10 of the sphygmomanometer 1A with the pressure generation pattern optimum for diagnosing the operation performance of the components to be diagnosed by the above-described method. If the measurement accuracy of the sphygmomanometer 1A is determined as abnormal, the causes that lower the measurement accuracy can be specified by diagnosing the operation performance on each component.

The embodiments disclosed herein are illustrative in all aspects and should not be construed as being restrictive. The scope of the invention is defined by the Claims rather than by the description made above, and meanings equivalent to the Claims and all modifications within the scope are intended to be encompassed therein.

### INDUSTRIAL APPLICABILITY

The present invention is applicable to a sphygmomanometer, and a measurement accuracy check system of the sphygmomanometer.

1. A measurement accuracy check system of a sphygmomanometer, comprising:
a sphygmomanometer having a blood pressure measurement mode for measuring a blood pressure based on a change in internal pressure of a cuff attached to a blood pressure measurement site, and an accuracy check mode for checking measurement accuracy in the blood pressure measurement mode; and
an accuracy check device communicably connected with the sphygmomanometer for determining the measurement accuracy of the sphygmomanometer in the accuracy check mode, wherein
the sphygmomanometer includes:
an air system piping communicating to the cuff in the blood pressure measurement mode and communicating to an air system of the accuracy check device in the accuracy check mode;
a pressurization and depressurization unit for adjusting pressure to be applied to the air system piping; and
a first pressure detection unit for detecting pressure in the air system piping,
the accuracy check device includes:
a pressure generator for generating pressure in the air system according to a predetermined pressure generation pattern set in advance; and
a second pressure detection unit for detecting pressure in the air system, and
one of the sphygmomanometer and the accuracy check device includes:
a measurement accuracy determining portion for determining the measurement accuracy of the sphygmomanometer based on a difference value between a pressure detection value of the first pressure detection unit and a pressure detection value of the second pressure detection unit ; and
a display unit for displaying the determined measurement accuracy of the sphygmomanometer.

2. The measurement accuracy check system of the sphygmomanometer according to claim 1, wherein the predetermined pressure generation pattern includes a pulse wave generation pattern for expressing a change in pulse pressure detected by the first pressure detection unit in the blood pressure measurement mode.

3. The measurement accuracy check system of the sphygmomanometer according to claim 2, wherein
the predetermined pressure generation pattern further includes a generation pattern for applying pressure to the air system piping for a predetermined period set in advance, and
the measurement accuracy determining portion includes an operation performance diagnosis portion for diagnosing operation performance of a component of the pressurization and depressurization unit based on the pressure detection value of the first pressure detection unit after elapse of the predetermined period.

4. The measurement accuracy check system of the sphygmomanometer according to claim 1, wherein
the sphygmomanometer further includes:
a storage unit for storing the determined measurement accuracy of the sphygmomanometer in association with check date and time of the measurement accuracy; and
a notifying unit for notifying whether or not the blood pressure measurement mode is executable on the user based on the measurement accuracy stored in the storage unit.

5. The measurement accuracy check system of the sphygmomanometer according to claim 4, wherein the notifying unit notifies to urge execution of the accuracy check mode to the user in association with the notification.

6. The measurement accuracy check system of the sphygmomanometer according to claim 1, wherein the sphygmomanometer further includes an operation unit for outputting a signal for instructing selection of the accuracy check mode in response to an operation by the user.

7. The measurement accuracy check system of the sphygmomanometer according to claim 1, wherein the sphygmomanometer further includes a connector for coupling the air system piping and an air system of the accuracy check device, the accuracy check mode being selected in response to closing of the connector.

8. A sphygmomanometer having a blood pressure measurement mode for measuring a blood pressure based on a change in internal pressure of a cuff attached to a blood pressure measurement site, and an accuracy check mode for checking measurement accuracy in the blood pressure measurement mode, the sphygmomanometer comprising:
an air system piping communicating to the cuff in the blood pressure measurement mode;
a pressurization and depressurization unit for adjusting pressure to be applied to the air system piping;
a first pressure detection unit for detecting pressure in the air system piping,
the air system piping communicating to an air system of an accuracy check device arranged outside the sphygmomanometer in the accuracy check mode, and being applied with pressure having a predetermined pressure generation pattern generated in the air system by the accuracy check device;
a measurement accuracy determining portion for determining the measurement accuracy of the sphygmomanometer based on a difference value between a pressure detection value of the first pressure detection unit and a predetermined pressure reference value set in advance in the accuracy check mode; and
a display unit for displaying the determined measurement accuracy of the sphygmomanometer.

9. The sphygmomanometer according to claim 8, further comprising a mode selection unit for detecting a pressure signal of the air system of the accuracy check device and selecting the accuracy check mode.

10. The measurement accuracy check system of the sphygmomanometer according to claim 2, wherein the sphygmomanometer further includes a connector for coupling the air system piping and an air system of the accuracy check device, the accuracy check mode being selected in response to closing of the connector.

11. The measurement accuracy check system of the sphygmomanometer according to claim 3, wherein the sphygmomanometer further includes a connector for coupling the air system piping and an air system of the accuracy check device, the accuracy check mode being selected in response to closing of the connector.

12. The measurement accuracy check system of the sphygmomanometer according to claim 4, wherein the sphygmomanometer further includes a connector for coupling the air system piping and an air system of the accuracy check device, the accuracy check mode being selected in response to closing of the connector.

13. The measurement accuracy check system of the sphygmomanometer according to claim 5, wherein the sphygmomanometer further includes a connector for coupling the air system piping and an air system of the accuracy check device, the accuracy check mode being selected in response to closing of the connector.

14. The measurement accuracy check system of the sphygmomanometer according to claim 2, wherein the sphygmomanometer further includes a connector for coupling the air system piping and an air system of the accuracy check device, the accuracy check mode being selected in response to closing of the connector.

15. The measurement accuracy check system of the sphygmomanometer according to claim 3, wherein the sphygmomanometer further includes a connector for coupling the air system piping and an air system of the accuracy check device, the accuracy check mode being selected in response to closing of the connector.

16. The measurement accuracy check system of the sphygmomanometer according to claim 4, wherein the sphygmomanometer further includes a connector for coupling the air system piping and an air system of the accuracy check device, the accuracy check mode being selected in response to closing of the connector.

17. The measurement accuracy check system of the sphygmomanometer according to claim 5, wherein the sphygmomanometer further includes a connector for coupling the air system piping and an air system of the accuracy check device, the accuracy check mode being selected in response to closing of the connector.
